(19) United States
(12) Patent Application Publication
(10) Pub. No.: US 2022/0184176 A1
(43) Pub. Date: Jun. 16, 2022

(54) AMPHIPHYSIN / BIN1 FOR THE TREATMENT OF AUTOSOMAL DOMINANT CENTRONUCLEAR MYOPATHY

(71) Applicants: CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR); UNIVERSITÉ DE STRASBOURG, STRASBOURG (FR)

(72) Inventors: VALENTINA MARIA LIONELLO, GERENZANO (IT); JOCELYN LAPORTE, STRASBOURG (FR)

(21) Appl. No.: 17/440,807

(22) PCT Filed: Mar. 20, 2020
(86) PCT No.: PCT/EP2020/057853

§ 371 Date: Sep. 20, 2021

Related U.S. Application Data
(60) Provisional application No. 62/820,932, filed on Mar. 20, 2019.

(51) Int. Cl. A61K 38/17; A61P 21/00

(52) U.S. Cl. CPC A61K 38/1709; A61P 21/00

(57) ABSTRACT

The present disclosure relates to a BIN1 protein or a BIN1 nucleic acid sequence producing or encoding the same, for a use in the treatment of Autosomal dominant centronuclear myopathy. The present invention provides compositions and methods for treatment of Autosomal dominant centronuclear myopathy. The present invention relates to a method of delivering the BIN1 polypeptide to subjects with Autosomal Dominant Centronuclear Myopathy.

### FIELD OF THE INVENTION

The present disclosure relates to a BIN1 protein or a BIN1 nucleic acid sequence producing or encoding the same, for a use in the treatment of Autosomal dominant centronuclear myopathy. The present invention provides compositions and methods for treatment of Autosomal dominant centronuclear myopathy. The present invention relates to a method of delivering the BIN1 polypeptide to subjects with Autosomal Dominant Centronuclear Myopathy.

### BACKGROUND OF THE INVENTION

Centronuclear Myopathies (CNM) are a group of congenital myopathies characterized by muscle weakness and confirmed histologically by fiber atrophy, predominance of type I fibers, and increased centralization of nuclei, not secondary to muscle regeneration. Among the three main characterized forms of CNM, the Autosomal Dominant Centronuclear myopathy (ADCNM) presents a severity of the condition and the associated signs and symptoms vary significantly among affected people. In people with a mild form, features of the condition generally don't develop until adolescence or early adulthood and may include slowly progressive muscle weakness, muscle pain with exercise and difficulty walking. Although some affected people will eventually lose the ability to walk, this usually does not occur before the 6th decade of life. In more severe cases, affected people may develop symptoms during infancy or early childhood such as hypotonia and generalized weakness. These children generally have delayed motor milestones and often need wheelchair assistance in childhood or adolescence.

Most cases of ADCNM are caused by mutations in the DNM2 gene. The condition is inherited in an autosomal dominant manner. Current treatment is based on alleviating the signs and symptoms present in each ADCNM patient, and may include physical and/or occupational therapy and assistive devices to help with mobility, eating and/or breathing.

Dynamins are large GTPase proteins that play important roles in membrane trafficking and endocytosis, and in actin cytoskeleton assembly. Dynamin proteins contain an N-terminal GTPase domain, middle domain, PH domain (phosphoinositide binding), GED (GTPase effector domain), and a PRD (Proline-rich domain) for protein-protein interactions. Three human dynamins have been identified to this day: dynamin 1, exclusively expressed in neurons; dynamin 3, predominantly expressed in brain and testis; and dynamin 2 (DNM2) which is ubiquitously expressed. DNM2 is a mechanoenzyme that is mainly implicated in vesicle budding in endocytosis and recycling and in cytoskeleton organization. Upon membrane binding, DNM2 oligomerizes around membrane tubules and its GTPase activity drives membrane fission.

In the case of ADCNM, previous studies have suggested that heterozygous DNM2 mutations are “gain-of-function” mutations, i.e. that they lead to an augmentation in DNM2 activities, without necessarily an increased in DNM2 expression level. DNM2-CNM mutations typically increase the DNM2 GTPase activity and oligomer stability in vitro. The most common mutation observed in ADCNM patients (DNM2 mutation in amino acid position 465, also named the R465W mutation) has notably been shown to favor DNM2 oligomerization. The creation and characterization of a knock-in mouse model carrying this mutation was previously conducted. Dnm2^{R465W/+} mice are viable and have a normal life span and body weight; they start to present muscle force and histological defects during the 2n^{d} month (Durieux et al., 2010 J Mol Med (Berl). 2010 April; 88(4):339-50. Doi: 10.1007/s00109-009-0587-4). Recently, Buono et al. (Buono et al., 2018 Proc Natl Acad Sci U S A. 2018 Oct. 23; 115(43):11066-11071. Doi: 10.1073/pnas.1808170115. Epub 2018 Oct 5.), proposed a novel therapeutic strategy to downregulating the total pool of DNM2 through oligonucleotide (ASO) or AAV-shRNA targeting the pre-mRNA and mRNA of DNM2 in Dnm2^{R465W/+} mice. These approaches allowed the rescue of skeletal muscle force and muscle histology and suggested that DNM2 is more active in Dnm2^{R465W/+} as the reduction of total protein level (not specific for mutated allele) rescued the CNM skeletal muscle phenotype.

However, these previous conducted studies focused on mice with heterozygous Dnm2 R465W mutation (mouse model for the late-onset ADCNM phenotype), because the homozygous mouse Dnm2^{R465W} (mouse model for the early-onset ADCNM phenotype) dies a few days after birth. Indeed, Durieux et al. 2010 observed that six homozygous Dnm2^{R465W/R465W} survived for 2 weeks after birth. Only one mouse was analyzed and showed an increase in connective tissue inside the muscle and reduced fiber size diameter compared to the WT control. The ultrastructure analysis showed a disorganization on the myofiber and an increase in tubular structure closed to the Z-line. No further investigations have been conducted on Dnm2 R465W/R465W mouse model. To date no study has presented a rescue in the life span of homozygous R465W/R465W mice.

BIN1 (i.e., Bridging Integrator 1) encodes for Amphiphysin 2 and mutations in this gene can cause CNM, and more particularly autosomal recessive CNM (also named ARCNM). BIN1 is ubiquitously expressed and it is essential for endocytosis, membrane recycling and remodeling. There are various tissue-specific isoforms of BIN1; among them, the skeletal muscle specific isoform is the isoform 8 which contains a phosphoinositides (PI) binding domain. This domain increases the affinity of BIN1 to the Ptdlns4,5P2, Ptdlns5P and Ptdlns3P. iln vitro studies have demonstrated the involvement of this phosphoinositides (PI) binding domain in the formation of membrane tubules that resemble the T tubule in skeletal muscle (Lee et al. Amphiphysin 2 (Bin1) and T-tubule biogenesis in muscle. Science. 2002 Aug. 16; 297(5584):1193-6. PMID:12183633).

Here, the present application demonstrates that overexpression of BIN1 is sufficient to rescue, or at least alleviate in the severe form, the ADCNM phenotype. In that regard, the Inventors discovered that BIN1 regulates DNM2 activity in skeletal muscle, in particular DNM2 oligomerization and membrane fission activity. Increasing BIN1 can ameliorate the pathophysiology in ADCNM mice models (Dnm2^{RW/+} and Dnm2^{RW/RW}) which makes BIN1 overexpression an effective therapy for the treatment of ADCNM in humans, at early or late onset of the disease.

### SUMMARY OF THE INVENTION

The present disclosure provides methods and compositions for treating ADCNM by overexpression of BIN1. The present invention provides compositions and methods for treatment of ADCNM, in a subject in need thereof.

The present invention relates to a method of expressing BIN1 to subjects with ADCNM. The compositions and methods of the present invention can increase muscle strength and/or improve muscle function and/or rescue histological features in a subject with ADCNM.

In one embodiment, the present invention is useful for treating an individual with ADCNM. In particular, the present invention relates to an Amphiphysin 2 polypeptide or a BIN1 nucleic acid sequence, for a use in the treatment of ADCNM. In other words, the invention relates to the use of an Amphiphysin 2 polypeptide or a BIN1 nucleic acid sequence, for the preparation of a medicament for the treatment ADCNM. More specifically, the invention relates to a method for treating ADCNM in a subject in need thereof, comprising administering to said subject a therapeutically effective amount of an Amphiphysin 2 polypeptide or a BIN1 nucleic acid sequence. Indeed, the present invention improves muscle function and prolongs survival in afflicted subjects.

In a particular aspect, the present invention concerns a composition comprising Amphiphysin 2 polypeptide or a nucleic acid sequence producing or encoding such polypeptide, such as BIN1. Said composition can be for use in the treatment of ADCNM.

The present invention also provides isolated polypeptides comprising Amphiphysin 2 protein, as well as pharmaceutical compositions comprising Amphiphysin 2 protein in combination with a pharmaceutical carrier.

The present invention also deals with an isolated nucleic acid sequence comprising at least one BIN1 nucleic acid sequence, or an expression vector comprising such nucleic acid sequence comprising at least one BIN1 nucleic acid sequence, as well as pharmaceutical compositions comprising the same in combination with a pharmaceutical carrier.

Further, the present invention relates to methods of making such Amphiphysin 2 or constructs comprising at least one BIN1 nucleic acid sequence.

Additionally, disclosed herein are methods of using Amphiphysin 2 polypeptide or expression vector comprising at least one BIN1 nucleic acid sequence, for the treatment of ADCNM.

These and other objects and embodiments of the invention will become more apparent after the detailed description of the invention.

### BRIEF DESCRIPTION OF THE DRAWINGS

FIG. 1: Characterization of Dnm2^{R465W/+} Tg BIN1 Mice (Dnm^{R465W/+} Mice Overexpressing BIN1)

(A) Western blot from Tibialis Anterior (TA) probed with anti BIN1 and DNM2 antibodies. (B) BIN1 quantification normalized to beta actin. Statistic test: Non parametric test for the graph B, Kruskall-Wallis post-hoc test. *p<0.05. (C) Lifespan represented as percentage of survival for WT, TgBIN1, Dnm2^{RW/+} and Dnm2^{RW/+} TgBIN1 mice. (D) Mouse body weight with age from 1 to 7 months (n≥5). (E), Hanging test: mice were suspended from a cage lid for maximum 60 s and each mouse repeated the test three times for each time point (n>5). (F-G) Rotarod test at 4 (F) and 8 months (G) of age.

FIG. 2: Overexpression of BIN1 in Dnm2 R465W /+ Improves In Situ Muscle Force

(A) TA muscle weight normalized on total body weight at 4 months (g/g). (B) Absolute maximal force of the TA at 4 and 8 months. (C) Specific TA muscle force at 4 and 8 months of age (n 7). Statistic test: One-way Anova and Bonferroni post-hoc test. *p<0.05, **p<0.01. Mean±SEM.

FIG. 3: Overexpressing BIN1 Ameliorates the Histopathology of Dnm2^{RW/+} Mice (Transversal TA Muscle Sections Stained with H&E and SDH):

(A) Transversal TA muscle sections stained with HE at 4 months. Scale bar: 100 μm. (B) Minimum ferret of TA fibers grouped into 5μm intervals at 4 months (n=3). Transversal TA muscle sections stained with NADH-TR (C) and SDH (D) at 4 and 8 months. (Arrows shows abnormal aggregates). Scale bar: 100 μm. Statistic test: Non parametric test for the graph B, Kruskall-Wallis post-hoc test. *p<0.05. Mean ±SEM. (E) Frequency of fibers with abnormal SDH staining at 4 and 8 months. (F) Longitudinal TA muscle ultrastructure observed by electron microscopy. Triads (arrowheads), longitudinal oriented T-tubule (arrow), enlarged mitochondria (star). Scale bar 0.5 μm. (G) High magnification view of the triads. Scale bar 0.1 μm. (H) Quantification of mis-oriented T-tubules (n≥2). (I) Cluster of enlarged mitochondria in Dnm2^{RW/+}: TA muscle ultrastructure observed by electron microscopy. Scale bar 1μm.

FIG. 4: Post-Natal Intramuscular Overexpression of BIN1 Improves the Histopathology of Dnm2^{RW/+} Mice

Dnm2^{RW/+} mice were injected at 3-weeks old with either AAV empty (AAV-Ctrl) in one leg or AAV-BIN1 in the contralateral leg and mice were analysed 4weeks post-injection (A) Western blot from Tibialis Anterior (TA) probed with anti-BIN1 and beta actinin antibodies. (B) Western blot quantification graph of BIN1 normalized on beta actinin. (C) TA muscle weight normalized on total body weight (g/g) (n≥3). (D) Absolute TA muscle force 4 weeks post intramuscular injection (n≥3). (E) Specific TA muscle force at 8 weeks old mice (n≥3). Statistic test: Non parametric test for the graph B, Kruskall-Wallis post-hoc test. *p<0.05. Mean±SEM. (F) Minimum ferret of TA fibers grouped into 5 μm intervals (n≥3). (G) Frequency of fibers with abnormal SDH staining.

FIG. 5: Post-Natal Intramuscular Overexpression of BIN1 Improves the Histopathology of Dnm2^{RW/+} Mice (Transversal TA Muscle Sections Stained with HE and SDH)

(A) Transversal TA muscle sections stained with HE. WT and Dnm2R465W/+ injected with AAV Ctrl and AAV-BIN1 isoform 8. (B-C) Transversal TA muscle sections stained with NADH-TR (B) and SDH (C). Dnm2R465W/+ muscles injected with AAV-CTRL have abnormal aggregates in the center of the fibers (arrow) which are not detectable in muscles injected with AAV-BIN1 isoform 8. Scale bar: 100 μm.

FIG. 6: BIN1 Overexpression Improves the Survival (i.e. Lifespan and Growth) of Dnm2 R465W/R465W Mice

(A) Mouse body weight with age (from 1 to 8 weeks) (n >5). (B), Hanging test at 2 months. Mice were suspended from a grid for maximum 60 seconds (n>5). (C), TA muscle weight normalized on total body weight (g/g) (n>5). (D) Absolute maximal TA muscle force at 8 weeks of age (n >5). (E), Specific maximal TA muscle force at 8 weeks of age (n =5). (F-G), Western blot from Tibialis Anterior (TA) probed with anti DNM2 and BIN1 antibodies. Quantification graph of DNM2 and BIN1 normalized to beta actin. (H) Percentage of survival for WT, Dnm2^{RW/RW} and Dnm2^{RW/RW} TgBIN1 mice. Statistic test: Non parametric test. Mann-Whitney post-hoc test. *p<0.05, **p<0.01, ***p<0.001.

FIG. 7: Dnm2R465W/R465W Tg BIN1 Muscle Histology and Structure

(A) Transversal TA muscle sections stained with HE. Scale bar 100 μm. (B) Minimum ferret of TA fibers grouped into 5μm intervals (n=5). (C) Frequency of muscle fibers with internalized nuclei (n=5). (D) Transversal TA muscle sections stained with SDH. Scale bar 100 μm. (E) Frequency of fibers with abnormal SDH staining (n=3). (F) TA muscle ultrastructure observed by electron microscopy. Scale bar 1μm. (G) Quantification of T-tubules roundness (n=2). (H) Transversal TA muscle section stained with a dysferlin antibody. Scale bar 10 μm. Statistic test: Student t-test *p<0.05, ** p<0.01, *** p<0.001.

FIG. 8: Characterization of BIN1-DNM2 Molecular Interaction

(A) Pull-down of DNM2 protein produced in insect cells with purified GST-BIN1 or GST-SH3 produced in bacteria. Coomassie staining. (B) Negative staining and electron microscopy of purified DNM2 and (C) purified DNM2 with BIN1. Scale bar 200 nm. Zoomed examples of DNM2 oligomers with or without BIN1: filament, horseshoe, ring (arrowheads) or ball (arrows). Scale bar 50 nm. (D) Quantification of the different DNM2 oligomers on a total of 678 structures counted. Statistic test: No parametric test Mann-Whitney test. Dunn's post hoc test *p<0.05, ** p<0.01, *** p<0.001. (E) BIN1 levels in Dnm2^{RW/RW} TgBIN1 mice: pull-down of DNM2 protein produced in insect cells with purified GST-SH3 (left panel) or GST-BIN1 (right panel) produced in bacteria. Coomassie staining.

FIG. 9: BIN1 and DNM2 Tubulation and Fission Activity

(A) Negative staining and electron microscopy of liposomes incubated with purified BIN1, DNM2+GTP, or BIN1+DNM2+GTP (1:1 ratio of BIN1:DNM2). Arrow points to a membrane tubule. Scale bar 200nm. (B) Quantification of the number of membrane tubules emanating from liposomes. (C) Quantification of liposomes diameter after incubation with DNM2+GTP or BIN1+DNM2+GTP (1:1 ratio of BIN1:DNM2); liposomes analyzed n>150. (D) COS-1 cells transfected with BIN1. (E)

Percentage of cells with BIN1 tubules after transfection with 0.5 or 1 μg of DNM2 WT or DNM2^{R465W} (n=3). Statistic test: No parametric test. Mann Whitney test and Student T-test: *p<0.05, **** p<0.0001. (F) COS-1 cells transfected with BIN1-GFP. (A) COS-1 cells transfected only with BIN1-GFP and probed anti DNM2.

### DETAILED DESCRIPTION

Unless otherwise defined, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“About” or “around” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods or compositions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

According to the invention, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated.

The terms “polypeptide,” “peptide” and “protein” are used interchangeably herein to refer to a polymer of amino acid residues covalently linked by peptide bonds. The terms apply to amino acid polymers in which one or more amino acid residue is an artificial chemical mimetic of a corresponding naturally occurring amino acid, as well as to naturally occurring amino acid polymers and non-naturally occurring amino acid polymers. “Polypeptides” include, for example, biologically active fragments, substantially homologous polypeptides, oligopeptides, homodimers, heterodimers, variants of polypeptides, modified polypeptides, derivatives, analogues, fusion proteins, among others. The polypeptides include natural peptides, recombinant peptides, synthetic peptides, or a combination thereof.

As used herein, “treating a disease or disorder” means reducing the frequency with which a symptom of the disease or disorder is experienced by a patient. Disease and disorder are used interchangeably herein. To “treat” a disease as the term is used herein, means to reduce the frequency or severity of at least one sign or symptom of a disease or disorder experienced by a subject. Within the context of the invention, the term treatment denotes curative, symptomatic, and preventive treatment. As used herein, the term “treatment” of a disease refers to any act intended to extend life span of subjects (or patients) such as therapy and retardation of the disease progression. The treatment can be designed to eradicate the disease, to stop the progression of the disease, and/or to promote the regression of the disease. The term “treatment” of a disease also refers to any act intended to decrease the symptoms associated with the disease, such as hypotonia and muscle weakness. More specifically, the treatment according to the invention is intended to delay the appearance of or revert ADCNM phenotypes or symptoms, ameliorate the motor and/or muscular behavior and/or lifespan.

A disease or disorder is “alleviated” if the severity of a symptom of the disease or disorder, the frequency with which such a symptom is experienced by a patient, or both, is reduced. A “therapeutic” treatment is a treatment administered to a subject who exhibits signs of pathology, for the purpose of diminishing or eliminating at least one or all of those signs.

In the present context, the disease to be treated is autosomal dominant centronuclear myopathy (ADCNM). ADCNM is associated with a wide-clinical spectrum of slowly progressive CNMs, from those beginning in childhood, adolescence/adulthood to more severe sporadic forms with neonatal onset. These different forms are characterized by multiple missense mutations in the DNM2 locus (chromosome 19 in humans), hence are also called DNM2-associated CNM (Böhm et al., Hum Mutat. 2012 June; 33(6):949-59. doi: 10.1002/humu.22067. Epub 2012 Apr 4. PMID: 22396310, incorporated herein by reference).

ADNCM can be divided into two subgroups due to the presence or absence of muscle hypertrophy: (i) classic form, also called mild form, which is characterized by late onset and slow progression, and (ii) with muscle hypertrophy, also called severe form, which is usually presents at a younger age and has a more rapid course.

In a preferred embodiment of the present invention, the autosomal-dominant centronuclear myopathy to be treated is a severe or mild form of ADCNM, preferably a mild form of ADCNM.

In a preferred embodiment of the present invention, the autosomal-dominant centronuclear myopathy is ADCNM at early onset or late onset, preferably at late onset. Early onset typically comprises neonatal onset, while late onset comprises childhood/adolescence or adult onset. Preferably, the ADNCM to be treated according to the invention is at childhood/adolescence or adult onset, more preferably at adult onset.

The phrase “therapeutically effective amount,” as used herein, refers to an amount that is sufficient or effective to prevent or treat (delay or prevent the onset of, prevent the progression of, inhibit, decrease or reverse) a disease or disorder, including provision of a beneficial effect to the subject or alleviating symptoms of such diseases.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. Preferably the subject is a human patient whatever its age or sex. Embryos, fetuses, new-borns (neonates), infants, children/adolescents are included as well. In the context of the present invention, ADCNM patients can be typically divided into neonates, children/adolescents and adults, as they display a different severity of the disease; the earlier the onset, the more severe the disease is. As demonstrated in the Examples, embryos and fetuses can also be treated according to the invention. Embryos and fetuses refer to unborn offspring; neonates typically encompass newborns from day 0 to about 1 year old, while childhood/adolescents can range from about 1-2 years old patients to about 16 years-old patients (included). Adults may accordingly comprise those aged over 16 years old.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed, which can be referred herein as a construct. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. The construct is therefore incorporated into an expression vector.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology. The “% of homology” between two nucleotide (or amino acid) sequences can be determined upon alignment of these sequences for optimal comparison. Optimal alignment of sequences may be herein preferably conducted by a global homology alignment algorithm should the alignment be performed using sequences of the same or similar length, such as by the algorithm described by Needleman and Wunsch (Journal of Molecular Biology; 1970, 48(3): 443-53), by computerized implementations of this algorithm (e.g., using the DNASTAR® Lasergene software), or by visual inspection. Alternatively, should the alignment be performed using sequences of distinct length, the optimal alignment of sequences can be preferably conducted by a local homology alignment algorithm, such as by the algorithm described by Smith and Waterson (Journal of Molecular Biology; 1981, 147: 195-197), by computerized implementations of this algorithm (e.g., using the DNASTAR® Lasergene software), or by visual inspection. Examples of global and local homology alignment algorithms are well-known to the skilled practitioner, and include, without limitation, ClustaIV (global alignment), ClustaIW (local alignment) and BLAST (local alignment).

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain (an) intron(s).

As used herein, the term “nucleic acid” or “polynucleotide” refers to a polymeric form of nucleotides of any length, either ribonucleotides or deoxyribonucleotides. Nucleic acids, nucleic acid sequences and polynucleotides as used herein are interchangeable. Thus, this term includes, but is not limited to, single-, double- or multi- stranded DNA or RNA, genomic DNA, cDNA, DNA-RNA hybrids, or a polymer comprising purine and pyrimidine bases, or other natural, chemically or biochemically modified, non-natural, or derived nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidatephosphodiester oligomer. The nucleic acid of the invention can be prepared by any method known to one skilled in the art, including chemical synthesis, recombination, and mutagenesis. In preferred embodiments, the nucleic acid of the invention is a DNA molecule, preferably a double stranded DNA molecule, and preferably synthesized by recombinant methods well known to those skilled in the art, such as the cloning of nucleic acid sequences from a recombinant library or a cell genome, using ordinary cloning technology and PCRTM, and the like, and by synthetic means.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The human BIN1 expression can rescue the myopathy displayed by Dnm2^{R465/+} mice, which makes it an effective agent for the treatment of ADCNM. This method can lead to sustained improvements in muscle strength, size, and function for ADCNM patients.

The human BIN1 gene is located from base pair 127048023 to base pair 127107400 on chromosome 2 NC_000002.12 location. The BIN1 gene or gene products are also known by other names, including but not limited to AMPH2, AMPHL, SH3P9. The cDNA BIN1 full length corresponds to the longest isoform found in human; it encompasses 19 exons. Said BIN1 sequence is represented by SEQ ID NO: 1, which does not contain the muscle specific exon 11 and is thus not naturally expressed in muscle. However, in the context of the present invention, the presence of exon 11 is not mandatory. While BIN1 has 20 exons in total on the DNA, these exons are never found all together at the RNA level in humans—though all 20 exons can be used according to the present invention. Parts of the sequence represented by SEQ ID NO: 1 or any combination of at least two or three different exons 1-20 of BIN1 (SEQ ID NO: 3-22, respectively), more preferably any combination of at least two or three different exons 1-20 of BIN1 (SEQ ID NO: 3-22, respectively) according to increasing numbering of exons 1-20, can be used according to the invention. The skilled person would readily understand that “according to the increasing number of exons” means that the exons are combined according to their sequential order, or in other words consecutive order. Preferably, the number of exons present in the BIN1 nucleic acid sequence of the invention is 3, 4, 5, 6, 7, 8, 9, 10, 11, 12, 13, 14, 15, 16, 17, 18, 19, or 20 exons selected from the 20 BIN1 exons represented by SEQ ID NO: 3-22, and more preferably according to an increasing numbering of said exons 1-20 within the sequence. For example, the following sequences can be used according to the invention: an artificial cDNA sequence comprising at least exons 1 to 6 and 8 to 11 (SEQ ID NO: 23), cDNA comprising at least exons 1 to 6, 8 to 10, 12, and 17 to 20 (SEQ ID NO: 25; also named long isoform 9), cDNA comprising at least exons 1 to 6, 8 to 10, 12, and 18 to 20 (SEQ ID NO: 31; also named short isoform 9), cDNA comprising at least exons 1 to 6, 8 to 12, and 18 to 20 (SEQ ID NO: 27; also named isoform 8—without exon 17, which is BIN1 short muscle isoform containing the muscle specific exon 11), or cDNA comprising at least exons 1 to 6, 8 to 12, and 17 to 20 (SEQ ID NO: 29; also named isoform 8—with exon 17, which is BIN1 long muscle isoform containing the muscle specific exon 11, and corresponds to the NCBI isoform 8). The BIN1 nucleic acid sequence used according to the invention is able to encode the amphiphysin 2 polypeptide of the present invention. Particularly preferred BIN1 nucleic acids according to the invention are cDNA comprising at least exons 1 to 6, 8 to 10, 12, and 17 to 20 (SEQ ID NO: 25), and cDNA comprising at least exons 1 to 6, 8 to 12, and 18 to 20 (SEQ ID NO: 27;).

As mentioned above, there are various tissue-specific isoforms or transcript variants of BIN1, among them, an isoform found in skeletal muscle specific is the isoform 8 which contains a phosphoinositides (PI) binding domain. Said cDNA isoform 8 is represented by SEQ ID NO: 27 or SEQ ID NO: 29, the corresponding proteins are represented by SEQ ID NO: 28 or SEQ ID NO: 30.

The natural human Amphiphysin 2 protein of the present invention is of 593 amino acids length. It is encoded by BIN1 gene (Gene ID 274). The Amphiphysin 2 protein is also known by other names, including but not limited to BIN1, AMPH2, AMPHL, SH3P9. Said protein is represented by SEQ ID NO: 2. As mentioned above, there are various tissue-specific isoforms of BIN1 gene. Parts of the sequence represented by SEQ ID NO: 2 or any polypeptide sequence deriving from or encoded by any combination of at least two or three different BIN1 exons 1-20, more preferably deriving from or encoded by any combination of at least two or three different BIN1 exons 1-20 (SEQ ID NO: 3-22, respectively) according to increasing numbering of BIN1 exons 1-20, can be used according to the invention. According to specific embodiments, the amphiphysin 2 polypeptide useful for the treatment of ADCNM comprises an amino acid sequence represented by SEQ ID NO: 2, 24, 26, 28, 30 or 32. Particularly preferred amphiphysin 2 polypeptides according to the invention comprise an amino acid sequence represented by SEQ ID NO:26 or 28.

In one aspect, the Amphiphysin 2 protein disclosed herein comprises an amino acid sequence at least 90% identical (or homologous) to SEQ ID NO: 2, 24, 26, 28, 30 or 32, or a bioactive fragment or variant thereof. In some embodiments, the Amphiphysin 2 comprises an amino acid sequence at least 80%, 85%, 90%, 95%, 97%, 98%, 99% or 100% identical to SEQ ID NO: 2, 24, 26, 28, 30 or 32, and is or less than 593 amino acids length, or a bioactive fragment or variant thereof.

As used herein, the Amphiphysin 2 disclosed herein can include various isoforms, fragments, variants, fusion proteins, and modified forms of the naturally occurring protein of the human Amphiphysin 2 which is of 593 amino acids length, as described above, and represented by SEQ ID NO:.2. Such isoforms, fragments or variants, fusion proteins, and modified forms of the naturally occurring Amphiphysin 2 polypeptide have at least a portion of the amino acid sequence of substantial sequence identity to the naturally occurring polypeptide, and retain at least one function of the naturally occurring Amphiphysin 2 polypeptide.

In certain embodiments, a bioactive fragment, variant, or fusion protein of the naturally occurring Amphiphysin 2 polypeptide comprises an amino acid sequence that is at least 80%, 85%, and preferably at least 90%, 95%, 97%, 98%, 99% or 100% identical to the naturally occurring Amphiphysin 2 of SEQ ID NO: 2, 26, 28, 30 or 32. As used herein, “fragments” or “variants” are understood to include bioactive fragments or bioactive variants that exhibit “bioactivity” as described herein. That is, bioactive fragments or variants of Amphiphysin 2 exhibit bioactivity that can be measured and tested. For example, bioactive fragments or variants exhibit the same or substantially the same bioactivity as native (i.e., wild-type, or normal) Amphiphysin 2 protein, and such bioactivity can be assessed by the ability of the fragment or variant to, e.g., curve or remodel membrane in vitro, upon transfection in cells, or in vivo, or bind known effector proteins, as dynamin 2, or lipids, as phosphoinositides. Methods in which to assess any of these criteria are described herein and/or one must refer more specifically to the following references: Amphiphysin 2 (Bin1) and T-tubule biogenesis in muscle. Lee E, Marcucci M, Daniell L, Pypaert M, Weisz O A, Ochoa G C, Farsad K, Wenk M R, De Camilli P. Science. 2002 Aug 16;297(5584):1193-6. PMID:12183633; Regulation of Bin1 SH3 domain binding by phosphoinositides. Kojima C, Hashimoto A, Yabuta I, Hirose M, Hashimoto S, Kanaho Y, Sumimoto H, Ikegami T, Sabe H. EM BO J. 2004 Nov. 10; 23(22):4413-22. Epub 2004 Oct 14. PMID: 15483625; Mutations in amphiphysin 2 (BIN1) disrupt interaction with dynamin 2 and cause autosomal recessive centronuclear myopathy. Nicot A S, Toussaint A, Tosch V, Kretz C, Wallgren-Pettersson C, lwarsson E, Kingston H, Garnier J M, Biancalana V, Oldfors A, Mandel J L, Laporte J. Nat Genet. 2007 Sep;39(9):1134-9. Epub 2007 Aug. 5.

In the context of the present invention, the function (or bioactivity) of Amphiphysin 2 polypeptide, or bioactive fragments or variants thereof, can also be tested as described in the Examples described below, notably by assessing e.g. improvement of survival, lifespan, muscle strength, coordination, organization of muscle fibers/muscle ultrastructure, focal adhesion, and/or DNM2 activity (GTPase activity, oligomerization, membrane fission/tubulation).

As used herein, “substantially the same” refers to any parameter (e.g., activity or bioactivity as described above) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%, 105%, or 110% of a control against which the parameter is measured.

In certain embodiments, any of the Amphiphysin 2 polypeptides disclosed herein are possibly for use in a chimeric polypeptide further comprising one or more polypeptide portions that enhance one or more of in vivo stability, in vivo half-life, uptake/administration, and/or purification.

As used herein, BIN1 nucleic acid sequence can include BIN1 nucleic acid sequence that encodes a protein or fragment of the invention (such as those mentioned above) and/or contains SEQ ID NO:1, 23, 25, 27, 29 or 31, or a fragment thereof. In one embodiment, the BIN1 nucleic acid sequence which can be used according to the invention hybridizes to the sequence of SEQ ID NO:1, 23, 25, 27, 29 or 31 under stringent conditions. In another embodiment, the invention provides a nucleic acid sequence complementary to the nucleic acid sequence of SEQ ID NO:1, 23, 25, 27, 29 or 31. In still another embodiment, the invention provides a nucleic acid sequence encoding a fusion protein of the invention. In a further embodiment, the invention provides an allelic variant of any of the BIN1 nucleic acid sequences of the invention.

The present invention provides a composition that increases BIN1 expression in a muscle. For example, in one embodiment, the composition comprises an isolated BIN1 nucleic acid sequence or a nucleic acid comprising at least one BIN1 nucleic acid sequence. As described herein, delivery of a composition comprising such nucleic acid sequence improves muscle function. Furthermore, the delivery of a composition comprising such nucleic acid sequence prolongs survival of a subject with ADCNM.

The present invention also concerns a pharmaceutical composition comprising an Amphiphysin 2 polypeptide as defined above, or expression vector comprising at least one BIN1 nucleic acid sequence as defined above, in combination with a pharmaceutical carrier. Also disclosed said compositions are for use in the treatment of ADCNM.

The present invention further concerns a method for the treatment of ADCNM, wherein the method comprises a step of administering into a subject in need of such treatment a therapeutically efficient amount of Amphiphysin 2 polypeptide, or expression vector comprising at least one BIN1 nucleic acid sequence, as defined above.

Finally, the present invention concerns the use of Amphiphysin 2 polypeptide, or expression vector comprising at least one BIN1 nucleic acid sequence, as defined above, for the preparation of a pharmaceutical composition for the treatment of ADCNM.

The isolated nucleic acid sequence or a biologically functional fragment or variant thereof as defined above can be obtained using any of the many recombinant methods known in the art, such as, for example by screening cDNA or DNA libraries from cells expressing the BIN1 gene, by deriving the gene from a vector known to include the same, or by isolating directly from cells and tissues containing the same, using standard techniques (such as PCR). Alternatively, the gene of interest can be produced synthetically, rather than cloned.

The present invention also includes a vector in which the isolated BIN1 nucleic acid sequence or the nucleic acid comprising at least one BIN1 nucleic acid sequence of the present invention is inserted; and which is generally operably linked to one or more control sequences that direct expression of BIN1. The art is replete with suitable vectors that are useful in the present invention. It also refers to a nucleic acid construct or a recombinant host cell transformed with the vector of the invention.

In summary, the expression of BIN1 nucleic acid sequence is typically achieved by operably linking a BIN1 nucleic acid sequence or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for gene therapy, using standard gene delivery protocols. Methods for gene delivery are known in the art. See, e.g., U.S. Pat. Nos. 5,399,346; 5,580,859; or 5,589,466. In another embodiment, the invention provides a gene therapy vector.

The BIN1 nucleic acid sequence of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. In a preferred embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In one embodiment, the BIN1 nucleic acid sequence is contained within an AAV vector. More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of myotubularin nucleic acid sequences, for minimizing immunogenicity, for tuning stability and particle lifetime, for efficient degradation, for accurate delivery to the nucleus, etc.

Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress. Other useful AAV serotypes include AAV1, AAV3, AAV4, AAV5, AAV6, AAV7, AAV8, AAV9, AAV10, as well as AAV-DJ and AAV-PHP.S.

In one embodiment, the vectors useful in the compositions and methods described herein contain, at a minimum, sequences encoding a selected AAV serotype capsid, e.g., an AAV8 capsid, or a fragment thereof. In another embodiment, useful vectors contain, at a minimum, sequences encoding a selected AAV serotype rep protein, e.g., AAV8 rep protein, or a fragment thereof. Optionally, such vectors may contain both AAV cap and rep proteins.

The AAV vectors of the invention may further contain a minigene comprising a BIN1 nucleic acid sequence as described above which is flanked by AAV 5′ (inverted terminal repeat) ITR and AAV 3′ ITR. A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a BIN1 nucleic acid sequence, or biologically functional fragment thereof; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In specific embodiments, such a stable host cell will contain the required component(s) under the control of a constitutive promoter. In other embodiments, the required component(s) may be under the control of an inducible promoter. Examples of suitable inducible and constitutive promoters are provided elsewhere herein, and are well known in the art. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the invention may be delivered to the packaging host cell in the form of any genetic element which transfers the sequences carried thereon. The selected genetic element may be delivered using any suitable method, including those described herein and any others available in the art. The methods used to construct any embodiment of this invention are known to those with skill in nucleic acid manipulation and include genetic engineering, recombinant engineering, and synthetic techniques. Similarly, methods of generating rAAV virions are well known and the selection of a suitable method is not a limitation on the present invention.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, as well as AAV-DJ and AAV-PHP.S or other known or as yet unknown AAV serotypes. These ITRs or other AAV components may be readily isolated from an AAV serotype using techniques available to those of skill in the art. Such an AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene is composed of, at a minimum, a BIN1 nucleic acid sequence (the transgene) and its regulatory sequences, and 5′ and 3′ AAV inverted terminal repeats (ITRs). In one embodiment, the ITRs of AAV serotype 2 are used. However, ITRs from other suitable serotypes may be selected. It is this minigene which is packaged into a capsid protein and delivered to a selected host cell. The BIN1 encoding nucleic acid coding sequence is operatively linked to regulatory components in a manner which permits transgene transcription, translation, and/or expression in a host cell.

In addition to the major elements identified above for the minigene, the AAV vector generally includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized. Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. Depending on the promoter, it appears that individual elements can function either cooperatively or independently to activate transcription.

In order to assess the expression of BIN1, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the composition comprises a naked isolated BIN1 nucleic acid as defined above, wherein the isolated nucleic acid is essentially free from transfection-facilitating proteins, viral particles, liposomal formulations and the like. It is well known in the art that the use of naked isolated nucleic acid structures, including for example naked DNA, works well with inducing expression in muscle. As such, the present invention encompasses the use of such compositions for local delivery to the muscle and for systemic administration (Wu et al., 2005, Gene Ther, 12(6): 477-486).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

For use in vivo, the nucleotides of the invention may be stabilized, via chemical modifications, such as phosphate backbone modifications (e.g., phosphorothioate bonds). The nucleotides of the invention may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors, or in combination with a cationic peptide. They can also be coupled to a biomimetic cell penetrating peptide. They may also be administered in the form of their precursors or encoding DNAs.

Chemically stabilized versions of the nucleotides also include “Morpholinos” (phosphorodiamidate morpholino oligomers—PMO), 2′-O-Methyl oligomers, AcHN-(RXRRBR)2XB peptide-tagged PMO (R, arginine, X, 6-aminohexanoic acid and B, ®-alanine) (PPMO), tricyclo-DNAs, or small nuclear (sn) RNAs. All these techniques are well known in the art. These versions of nucleotides could also be used for exon skipping to promote expression of endogenous BIN1.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

Regardless of the method used to introduce exogenous nucleic acids into a host cell or otherwise expose a cell to the BIN1 nucleic acid sequence of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Genome editing can also be used as a tool according to the invention. Genome editing is a type of genetic engineering in which DNA is inserted, replaced, or removed from a genome using artificially engineered nucleases, or “molecular scissors”. The nucleases create specific double-stranded break (DSBs) at desired locations in the genome, and harness the cell's endogenous mechanisms to repair the induced break by natural processes of homologous recombination (HR) and non-homologous end-joining (NHEJ). There are currently four families of engineered nucleases being used: Zinc finger nucleases (ZFNs), Transcription Activator-Like Effector Nucleases (TALENs), the CRISPR/Cas system (more specifically Cas9 system, as described by P. Mali et al., in Nature Methods, vol. 10 No. 10, October 2013), or engineered meganuclease re-engineered homing endonucleases. Said nucleases can be delivered to the cells either as DNAs or mRNAs, such DNAs or mRNAs are engineered to overexpress BIN1 according to the invention. The CRISPR/Cas system can be used, in fusion with activator or regulator proteins to enhance expression of BIN1 through transcriptional activation or epigenetic modification (Vora S, Tuttle M, Cheng J, Church G, FEBS J. 2016 September; 283(17):3181-93. doi: 10.1111/febs.13768. Epub 2016 Jul 2. Next stop for the CRISPR revolution: RNA-guided epigenetic regulators).

The nucleotides as defined above used according to the invention can be administered in the form of DNA precursors.

The Amphiphysin 2 polypeptide as defined above, including fragments or variants thereof, can be chemically synthesized using techniques known in the art such as conventional solid phase chemistry. The fragments or variants can be produced (by chemical synthesis, for instance) and tested to identify those fragments or variants that can function as well as or substantially similarly to the native protein, for example, by testing their ability to curve or remodel membrane in vitro, upon transfection in cells, or in vivo, or bind known effector proteins, as dynamin 2, or lipids, as phosphoinositides, or treat ADCNM.

In certain embodiments, the present invention contemplates modifying the structure of an amphiphysin 2 polypeptide for such purposes as enhancing therapeutic or prophylactic efficacy, or stability (e.g., ex vivo shelf life and resistance to proteolytic degradation in vivo). Such modified amphiphysin 2 polypeptides have the same or substantially the same bioactivity as naturally-occurring (i.e., native or wild-type) amphiphysin 2 polypeptide. Modified polypeptides can be produced, for instance, by amino acid substitution, deletion, or addition at one or more positions. For instance, it is reasonable to expect, for example, that an isolated replacement of a leucine with an isoleucine or valine, an aspartate with a glutamate, or a similar replacement of an amino acid with a structurally related amino acid (e.g., conservative mutations) will not have a major effect on the biological activity of the resulting molecule. Conservative replacements are those that take place within a family of amino acids that are related in their side chains.

In a particular embodiment, the therapeutically effective amount to be administered according to the invention is an amount sufficient to alleviate at least one or all of the signs of ADCNM, or to improve muscle function of subject with ADCNM. The amount of amphiphysin 2 or of expression vector comprising at least one BIN1 nucleic acid sequence to be administered can be determined by standard procedure well known by those of ordinary skill in the art. Physiological data of the patient (e.g. age, size, and weight), the routes of administration and the disease to be treated have to be taken into account to determine the appropriate dosage, optionally compared with subjects that do not present centronuclear myopathies. One skilled in the art will recognize that the amount of amphiphysin 2 polypeptide or of a vector containing comprising at least one BIN1 nucleic acid sequence to be administered will be an amount that is sufficient to treat at least one or all of the signs of ADCNM, or to improve muscle function of subject with ADCNM. Such an amount may vary inter alia depending on such factors as the selected amphiphysin 2 polypeptides or vector expressing the same or expression vectors comprising at least one BIN1 nucleic acid sequence polypeptide, the gender, age, weight, overall physical condition of the patient, etc. and may be determined on a case by case basis. The amount may also vary according to other components of a treatment protocol (e.g. administration of other pharmaceuticals, etc.). Generally, when the therapeutic agent is a nucleic acid, a suitable dose is in the range of from about 1 mg/kg to about 100 mg/kg, and more usually from about 2 mg/kg/day to about 10 mg/kg. If a viral-based delivery of the nucleic acid is chosen, suitable doses will depend on different factors such as the virus that is employed, the route of delivery (intramuscular, intravenous, intra-arterial or other), but may typically range from 10-9 to 10-15 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient may be a single event, or the patient is administered with the amphiphysin 2 or nucleic acid encoding the same or expression vector comprising at least one BIN1 nucleic acid sequence on multiple occasions, that may be, depending on the results obtained, several days apart, several weeks apart, or several months apart, or even several years apart.

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, rectal, intravaginal, mucosal, topical (including transdermal, buccal and sublingual), or parenteral (including subcutaneous (sc), intramuscular (im), intravenous (iv), intra-arterial, intradermal, intrasternal, injection, or infusion techniques) administration. For these formulations, conventional excipient can be used according to techniques well known by those skilled in the art.

In particular, intramuscular or systemic administration is preferred. More particularly, in order to provide a localized therapeutic effect, specific muscular or intramuscular administration routes are preferred.

Pharmaceutical compositions according to the invention may be formulated to release the active drug substantially immediately upon administration or at any predetermined time or time period after administration.

SEQUENCE LISTING
[cDNA HUMAN BIN1 isoform 1 (longest BIN1 isoform)]
SEQ ID NO: 1
ATGGCAGAGATGGGCAGTAAAGGGGTGACGGCGGGAAAGATCGCCAGCAACGTGCAGAAGAAGCTCACCCG
CGCGCAGGAGAAGGTTCTCCAGAAGCTGGGGAAGGCAGATGAGACCAAGGATGAGCAGTTTGAGCAGTGCG
TCCAGAATTTCAACAAGCAGCTGACGGAGGGCACCCGGCTGCAGAAGGATCTCCGGACCTACCTGGCCTCCGT
CAAAGCCATGCACGAGGCTTCCAAGAAGCTGAATGAGTGTCTGCAGGAGGTGTATGAGCCCGATTGGCCCGG
CAGGGATGAGGCAAACAAGATCGCAGAGAACAACGACCTGCTGTGGATGGATTACCACCAGAAGCTGGTGGA
CCAGGCGCTGCTGACCATGGACACGTACCTGGGCCAGTTCCCCGACATCAAGTCACGCATTGCCAAGCGGGGG
CGCAAGCTGGTGGACTACGACAGTGCCCGGCACCACTACGAGTCCCTTCAAACTGC
CAAAAAGAAGGATGAAGCCAAAATTGCCAAGCCTGTCTCGCTGCTTGAGAAAGCCGCCCCCCAGTGGTGCCAA
GGCAAACTGCAGGCTCATCTCGTAGCTCAAACTAACCTGCTCCGAAATCAGGCCGAGGAGGAGCTCATCAAAG
CCCAGAAGGTGTTTGAGGAGATGAATGTGGATCTGCAGGAGGAGCTGCCGTCCCTGTGGAACAGCCGCGTAG
GTTTCTACGTCAACACGTTCCAGAGCATCGCGGGCCTGGAGGAAAACTTCCACAAGGAGATGAGCAAGCTCAA
CCAGAACCTCAATGATGTGCTGGTCGGCCTGGAGAAGCAACACGGGAGCAACACCTTCACGGTCAAGGCCCA
GCCCAGTGACAACGCGCCTGCAAAAGGGAACAAGAGCCCTTCGCCTCCAGATGGCTCCCCTGCCGCCACCCCC
GAGATCAGAGTCAACCACGAGCCAGAGCCGGCCGGCGGGGCCACGCCCGGGGCCACCCTCCCCAAGTCCCCA
TCTCAGCTCCGGAAAGGCCCACCAGTCCCTCCGCCTCCCAAACACACCCCGTCCAAGGAAGTCAAGCAGGAGC
AGATCCTCAGCCTGTTTGAGGACACGTTTGTCCCTGAGATCAGCGTGACCACCCCCTCCCAGTTTGAGGCCCCG
GGGCCTTTCTCGGAGCAGGCCAGTCTGCTGGACCTGGACTTTGACCCCCTCCCGCCCGTGACGAGCCCTGTGA
AGGCACCCACGCCCTCTGGTCAGTCAATTCCATGGGACCTCTGGGAGCCCACAGAGAGTCCAGCCGGCAGCCT
GCCTTCCGGGGAGCCCAGCGCTGCCGAGGGCACCTTTGCTGTGTCCTGGCCCAGCCAGACGGCCGAGCCGGG
GCCTGCCCAACCAGCAGAGGCCTCGGAGGTGGCGGGTGGGACCCAACCTGCGGCTGGAGCCCAGGAGCCAG
GGGAGACGGCGGCAAGTGAAGCAGCCTCCAGCTCTCTTCCTGCTGTCGTGGTGGAGACCTTCCCAGCAACTGT
GAATGGCACCGTGGAGGGCGGCAGTGGGGCCGGGCGCTTGGACCTGCCCCCAGGTTTCATGTTCAAGGTACA
GGCCCAGCACGACTACACGGCCACTGACACAGACGAGCTGCAGCTCAAGGCTGGTGATGTGGTGCTGGTGAT
CCCCTTCCAGAACCCTGAAGAGCAGGATGAAGGCTGGCTCATGGGCGTGAAGGAGAGCGACTGGAACCAGCA
CAAGGAGCTGGAGAAGTGCCGTGGCGT CTTCCCCGAGAACTTCACTGAGAGGGTCCCATGA
[AMINO ACID SEQUENCE of HUMAN BIN1 isoform 1 (longest BIN1 isoform)]
SEQ ID NO: 2
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKPVSLLEKAAPQWCQGKLQAHLVAQTNLLRNQAEEELIKAQKVFEEMNVDL
QEELPSLWNSRVGFYVNTFQSIAGLEENFHKEMSKLNQNLNDVLVGLEKQHGSNTFTVKAQPSDNAPAKGNKSPS
PPDGSPAATPEIRVNHEPEPAGGATPGATLPKSPSQLRKGPPVPPPPKHTPSKEVKQEQILSLFEDTFVPEISVTTPSQ
FEAPGPFSEQASLLDLDEDPLPPVTSPVKAPTPSGQSIPWDLWEPTESPAGSLPSGEPSAAEGTFAVSWPSQTAEPG
PAQPAEASEVAGGTQPAAGAQEPGETAASEAASSSLPAVVVETFPATVNGTVEGGSGAGRLDLPPGEMFKVQAQ
HDYTATDTDELQLKAGDVVLVIPFQNPEEQDEGWLMGVKESDWNQHKELEKCRGVFPENFTERVP
[BIN1 EXON 1]
SEQ ID NO: 3
Atggcagagatgggcagtaaaggggtgacggcgggaaagatcgccagcaacgtgcagaagaagctcacccgcgcgcaggagaag
[BIN1 EXON 2]
SEQ ID NO: 4
Gttctccagaagctggggaaggcagatgagaccaaggatgagcagtttgagcagtgcgtccagaatttcaacaagcagctg
[BIN1 EXON 3]
SEQ ID NO: 5
acggagggcacccggctgcagaaggatctccggacctacctggcctccgtcaaag
[BIN 1 EXON 4]
SEQ ID NO: 6
Ccatgcacgaggcttccaagaagctgaatgagtgtctgcaggaggtgtatgagcccgattggcccggcagggatgaggcaaacaagatcgcag
ag
[BIN1 EXON 5]
SEQ ID NO: 7
Aacaacgacctgctgtggatggattaccaccagaagctggtggaccaggcgctgctgaccatggacacgtacctgggccagttccccgacatca
ag
[BIN1 EXON 6]
SEQ ID NO: 8
Tcacgcattgccaagcgggggcgcaagctggtggactacgacagtgcccggcaccactacgagtcccttcaaactgccaaaaagaaggatgaa
gccaaaattgccaag
[BIN1 EXON 7, not present in skeletal muscle isoform]
SEQ ID NO: 9
Cctgtctcgctgcttgagaaagccgccccccagtggtgccaaggcaaactgcaggctcatctcgtagctcaaactaacctgctccgaaatcag
[BIN1 EXON 8]
SEQ ID NO: 10
Gccgaggaggagctcatcaaagcccagaaggtgtttgaggagatgaatgtggatctgcaggaggagctgccgtccctgtggaacag
[BIN1 EXON 9]
SEQ ID NO: 11
Ccgcgtaggtttctacgtcaacacgttccagagcatcgcgggcctggaggaaaacttccacaaggagatgagcaag
[BIN1 EXON 10]
SEQ ID NO: 12
Ctcaaccagaacctcaatgatgtgctggtcggcctggagaagcaacacgggagcaacaccttcacggtcaaggcccagcccag
[BIN1 EXON 11, muscle specific exon]
SEQ ID NO: 13
aaagaaaagtaaactgttttcgcggctgcgcagaaagaagaacag
[BIN1 EXON 12, not present in the skeletal muscle isoform]
SEQ ID NO: 14
tgacaacgcgcctgcaaaagggaacaagagcccttcgcctccagatggctcccctgccgccacccccgagatcagagtcaaccacgagccaga
gccggccggcggggccacgcccggggccaccctccccaagtccccatctcag
[BIN1 EXON 13, not present in skeletal muscle isoform]
SEQ ID NO: 15
ctccggaaaggcccaccagtccctccgcctcccaaacacaccccgtccaaggaagtcaagcaggagcagatcctcagcctgtttgaggacacgt
ttgtccctgagatc agcgtgaccaccccctcccag
[BIN 1 EXON 14, not present in skeletal muscle isoform]
SEQ ID NO: 16
tttgaggccccggggcctttctcggagcaggccagtctgctggacctggactttgaccccctcccgcccgtgacgagccctgtgaaggcacccacg
ccctctggtcag
[BIN 1 EXON 15, not present in skeletal muscle isoform]
SEQ ID NO: 17
tcaattccatgggacctctgggag
[BIN 1 EXON 16, not present in skeletal muscle isoform]
SEQ ID NO: 18
cccacagagagtccagccggcagcctgccttccggggagcccagcgctgccgagggcacctttgctgtgtcctggcccagccagacggccgagc
cggggcctgcccaa
ccagcagaggcctcggaggtggcgggtgggacccaacctgcggctggagcccaggagccaggggagacggcggcaagtgaagcagcctcc
[BIN 1 EXON 18]
SEQ ID NO: 20
Agctctcttcctgctgtcgtggtggagaccttcccagcaactgtgaatggcaccgtggagggcggcagtggggccgggcgcttggacctgccccc
aggtttcatgttcaag
[BIN1 EXON 19]
SEQ ID NO: 21
Gtacaggcccagcacgactacacggccactgacacagacgagctgcagctcaaggctggtgatgtggtgctggtgatccccttccagaaccctg
aagagcag
[BIN1 EXON 20]
SEQ ID NO: 22
gatgaaggctggctcatgggcgtgaaggagagcgactggaaccagcacaaggagctggagaagtgccgtggcgtcttccccgagaacttcact
gagagggtcccatga
[artificial cDNA sequence with BIN1 exons 1 to 6 and 8 to 11, corresponding to a
partial BIN1 isoform 8]
SEQ ID NO: 23
atggcagagatgggcagtaaaggggtgacggcgggaaagatcgccagcaacgtgcagaagaagctcacccgcgcgcaggagaaggttctcca
gaagctggggaaggcagatgagaccaaggatgagcagtttgagcagtgcgtccagaatttcaacaagcagctgacggagggcacccggctgca
gaaggatctccggacctacctggcctccgtcaaagccatgcacgaggcttccaagaagctgaatgagtgtctgcaggaggtgtatgagcccgatt
ggcccggcagggatgaggcaaacaagatcgcagagaacaacgacctgctgtggatggattaccaccagaagctggtggaccaggcgctgctga
ccatggacacgtacctgggccagttccccgacatcaagtcacgcattgccaagcgggggcgcaagctggtggactacgacagtgcccggcacca
ctacgagtcccttcaaactgccaaaaagaaggatgaagccaaaattgccaaggccgaggaggagctcatcaaagcccagaaggtgtttgagga
gatgaatgtggatctgcaggaggagctgccgtccctgtggaacagccgcgtaggtttctacgtcaacacgttccagagcatcgcgggcctggagg
aaaacttccacaaggagatgagcaagctcaaccagaacctcaatgatgtgctggtcggcctggagaagcaacacgggagcaacaccttcacgg
tcaaggcccagcccagaaagaaaagtaaactgttttcgcggctgcgcagaaagaagaacag
[AMINO ACID SEQUENCE of partial BIN1 isoform 8]
SEQ ID NO: 24
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKAEEELIKAQKVFEEMNVDLQEELPSLWNSRVGFYVNTFQSIAGLEENFHKEM
SKLNQNLNDVLVGLEKQHGSNTFTVKAQPRKKSKLFSRLRRKKNS
[cDNA sequence with BIN1 exons 1 to 6, 8 to 10, 12, and 17 to 20 - named BIN1
isoform 9]
SEQ ID NO: 25
atggcagagatgggcagtaaaggggtgacggcgggaaagatcgccagcaacgtgcagaagaagctcacccgcgcgcaggagaaggttctcca
gaagctggggaaggcagatgagaccaaggatgagcagtttgagcagtgcgtccagaatttcaacaagcagctgacggagggcacccggctgca
gaaggatctccggacctacctggcctccgtcaaagccatgcacgaggcttccaagaagctgaatgagtgtctgcaggaggtgtatgagcccgatt
ggcccggcagggatgaggcaaacaagatcgcagagaacaacgacctgctgtggatggattaccaccagaagctggtggaccaggcgctgctga
ccatggacacgtacctgggccagttccccgacatcaagtcacgcattgccaagcgggggcgcaagctggtggactacgacagtgcccggcacca
ctacgagtcccttcaaactgccaaaaagaaggatgaagccaaaattgccaaggccgaggaggagctcatcaaagcccagaaggtgtttgagga
gatgaatgtggatctgcaggaggagctgccgtccctgtggaacagccgcgtaggtttctacgtcaacacgttccagagcatcgcgggcctggagg
aaaacttccacaaggagatgagcaagctcaaccagaacctcaatgatgtgctggtcggcctggagaagcaacacgggagcaacaccttcacgg
tcaaggcccagcccagtgacaacgcgcctgcaaaagggaacaagagcccttcgcctccagatggctcccctgccgccacccccgagatcagagt
caaccacgagccagagccggccggcggggccacgcccggggccaccctccccaagtccccatctcagccagcagaggcctcggaggtggcggg
tgggacccaacctgcggctggagcccaggagccaggggagacggcggcaagtgaagcagcctccagctctcttcctgctgtcgtggtggagacc
ttcccagcaactgtgaatggcaccgtggagggcggcagtggggccgggcgcttggacctgcccccaggtttcatgttcaaggtacaggcccagca
cgactacacggccactgacacagacgagctgcagctcaaggctggtgatgtggtgctggtgatccccttccagaaccctgaagagcaggatgaa
ggctggctcatgggcgtgaaggagagcgactggaaccagcacaaggagctggagaagtgccgtggcgtcttccccgagaacttcactgagagg
gtcccatga
[AMINO ACID SEQUENCE of BIN1 isoform 9]
SEQ ID NO: 26
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKAEEELIKAQKVFEEMNVDLQEELPSLWNSRVGFYVNTFQSIAGLEENFHKEM
SKLNQNLNDVLVGLEKQHGSNTFTVKAQPSDNAPAKGNKSPSPPDGSPAATPEIRVNHEPEPAGGATPGATLPKSP
SQPAEASEVAGGTQPAAGAQEPGETAASEAASSSLPAVVVETFPATVNGTVEGGSGAGRLDLPPGFMFKVQAQH
DYTATDTDELQLKAGDVVLVIPFQNPEEQDEGWLMGVKESDWNQHKELEKCRGVFPENFTERVP
[cDNA with BIN1 exons 1 to 6, 8 to 12, and 18 to 20 - corresponding to BIN1 isoform
8 without exon 17, also named BIN1 short muscle isoform 13]
SEQ ID NO: 27
atggcagagatgggcagtaaaggggtgacggcgggaaagatcgccagcaacgtgcagaagaagctcacccgcgcgcaggagaaggttctcca
gaagctggggaaggcagatgagaccaaggatgagcagtttgagcagtgcgtccagaatttcaacaagcagctgacggagggcacccggctgca
gaaggatctccggacctacctggcctccgtcaaagccatgcacgaggcttccaagaagctgaatgagtgtctgcaggaggtgtatgagcccgatt
ggcccggcagggatgaggcaaacaagatcgcagagaacaacgacctgctgtggatggattaccaccagaagctggtggaccaggcgctgctga
ccatggacacgtacctgggccagttccccgacatcaagtcacgcattgccaagcgggggcgcaagctggtggactacgacagtgcccggcacca
ctacgagtcccttcaaactgccaaaaagaaggatgaagccaaaattgccaaggccgaggaggagctcatcaaagcccagaaggtgtttgagga
gatgaatgtggatctgcaggaggagctgccgtccctgtggaacagccgcgtaggtttctacgtcaacacgttccagagcatcgcgggcctggagg
aaaacttccacaaggagatgagcaagctcaaccagaacctcaatgatgtgctggtcggcctggagaagcaacacgggagcaacaccttcacgg
tcaaggcccagcccagaaagaaaagtaaactgttttcgcggctgcgcagaaagaagaacagtgacaacgcgcctgcaaaagggaacaagagc
ccttcgcctccagatggctcccctgccgccacccccgagatcagagtcaaccacgagccagagccggccggcggggccacgcccggggccaccc
tccccaagtccccatctcagagctctcttcctgctgtcgtggtggagaccttcccagcaactgtgaatggcaccgtggagggcggcagtggggccg
ggcgcttggacctgcccccaggtttcatgttcaaggtacaggcccagcacgactacacggccactgacacagacgagctgcagctcaaggctggt
gatgtggtgctggtgatccccttccagaaccctgaagagcaggatgaaggctggctcatgggcgtgaaggagagcgactggaaccagcacaag
gagctggagaagtgccgtggcgtcttccccgagaacttcactgagagggtcccatga
[AMINO ACID SEQUENCE of BIN1 isoform 13]
SEQ ID NO: 28
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKAEEELIKAQKVFEEMNVDLQEELPSLWNSRVGFYVNTFQSIAGLEENFHKEM
SKLNQNLNDVLVGLEKQHGSNTFTVKAQPRKKSKLFSRLRRKKNSDNAPAKGNKSPSPPDGSPAATPEIRVNHEPE
PAGGATPGATLPKSPSQSSLPAVVVETFPATVNGTVEGGSGAGRLDLPPGFMFKVQAQHDYTATDTDELQLKAGD
VVLVIPFQNPEEQDEGWLMGVKESDWNQHKELEKCRGVFPENFTERVP
[cDNA with BIN1 exons 1 to 6, 8 to 12, and 17 to 20: it is the BIN1 long muscle
isoform containing the muscle specific BIN1 exon 11 and also BIN1 exon 17, also
named BIN1 isoform 8]
SEQ ID NO: 29
atggcagagatgggcagtaaaggggtgacggcgggaaagatcgccagcaacgtgcagaagaagctcacccgcgcgcaggagaaggttctcca
gaagctggggaaggcagatgagaccaaggatgagcagtttgagcagtgcgtccagaatttcaacaagcagctgacggagggcacccggctgca
gaaggatctccggacctacctggcctccgtcaaagccatgcacgaggcttccaagaagctgaatgagtgtctgcaggaggtgtatgagcccgatt
ggcccggcagggatgaggcaaacaagatcgcagagaacaacgacctgctgtggatggattaccaccagaagctggtggaccaggcgctgctga
ccatggacacgtacctgggccagttccccgacatcaagtcacgcattgccaagcgggggcgcaagctggtggactacgacagtgcccggcacca
ctacgagtcccttcaaactgccaaaaagaaggatgaagccaaaattgccaaggccgaggaggagctcatcaaagcccagaaggtgtttgagga
gatgaatgtggatctgcaggaggagctgccgtccctgtggaacagccgcgtaggtttctacgtcaacacgttccagagcatcgcgggcctggagg
aaaacttccacaaggagatgagcaagctcaaccagaacctcaatgatgtgctggtcggcctggagaagcaacacgggagcaacaccttcacgg
tcaaggcccagcccagaaagaaaagtaaactgttttcgcggctgcgcagaaagaagaacagtgacaacgcgcctgcaaaagggaacaagagc
ccttcgcctccagatggctcccctgccgccacccccgagatcagagtcaaccacgagccagagccggccggcggggccacgcccggggccaccc
tccccaagtccccatctcagccagcagaggcctcggaggtggcgggtgggacccaacctgcggctggagcccaggagccaggggagacggcgg
caagtgaagcagcctccagctctcttcctgctgtcgtggtggagaccttcccagcaactgtgaatggcaccgtggagggcggcagtggggccggg
cgcttggacctgcccccaggtttcatgttcaaggtacaggcccagcacgactacacggccactgacacagacgagctgcagctcaaggctggtga
tgtggtgctggtgatccccttccagaaccctgaagagcaggatgaaggctggctcatgggcgtgaaggagagcgactggaaccagcacaagga
gctggagaagtgccgtggcgtcttccccgagaacttcactgagagggtcccatga
[AMINO ACID SEQUENCE of BIN1 isoform 8]
SEQ ID NO: 30
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKAEEELIKAQKVFEEMNVDLQEELPSLWNSRVGFYVNTFQSIAGLEENFHKEM
SKLNQNLNDVLVGLEKQHGSNTFTVKAQPRKKSKLFSRLRRKKNSDNAPAKGNKSPSPPDGSPAATPEIRVNHEPE
PAGGATPGATLPKSPSQPAEASEVAGGTQPAAGAQEPGETAASEAASSSLPAVVVETFPATVNGTVEGGSGAGRL
DLPPGFMFKVQAQHDYTATDTDELQLKAGDVVLVIPFQNPEEQDEGWLMGVKESDWNQHKELEKCRGVFPENF
TERVP
[artificial cDNA sequence with BIN1 exons 1 to 6; 8 to 10; 12 and 18-20 - named BIN1
isoform 10]
SEQ ID NO: 31
atggcagagatgggcagtaaaggggtgacggcgggaaagatcgccagcaacgtgcagaagaagctcacccgcgcgcaggagaaggttctcca
gaagctggggaaggcagatgagaccaaggatgagcagtttgagcagtgcgtccagaatttcaacaagcagctgacggagggcacccggctgca
gaaggatctccggacctacctggcctccgtcaaagccatgcacgaggcttccaagaagctgaatgagtgtctgcaggaggtgtatgagcccgatt
ggcccggcagggatgaggcaaacaagatcgcagagaacaacgacctgctgtggatggattaccaccagaagctggtggaccaggcgctgctga
ccatggacacgtacctgggccagttccccgacatcaagtcacgcattgccaagcgggggcgcaagctggtggactacgacagtgcccggcacca
ctacgagtcccttcaaactgccaaaaagaaggatgaagccaaaattgccaaggccgaggaggagctcatcaaagcccagaaggtgtttgagga
gatgaatgtggatctgcaggaggagctgccgtccctgtggaacagccgcgtaggtttctacgtcaacacgttccagagcatcgcgggcctggagg
aaaacttccacaaggagatgagcaagctcaaccagaacctcaatgatgtgctggtcggcctggagaagcaacacgggagcaacaccttcacgg
tcaaggcccagcccagtgacaacgcgcctgcaaaagggaacaagagcccttcgcctccagatggctcccctgccgccacccccgagatcagagt
caaccacgagccagagccggccggcggggccacgcccggggccaccctccccaagtccccatctcagagctctcttcctgctgtcgtggtggaga
ccttcccagcaactgtgaatggcaccgtggagggcggcagtggggccgggcgcttggacctgcccccaggtttcatgttcaaggtacaggcccag
cacgactacacggccactgacacagacgagctgcagctcaaggctggtgatgtggtgctggtgatccccttccagaaccctgaagagcaggatg
aaggctggctcatgggcgtgaaggagagcgactggaaccagcacaaggagctggagaagtgccgtggcgtcttccccgagaacttcactgaga
gggtcccatga
[AMINO ACID SEQUENCE of BIN1 isoform 10]
SEQ ID NO: 32
MAEMGSKGVTAGKIASNVQKKLTRAQEKVLQKLGKADETKDEQFEQCVQNFNKQLTEGTRLQKDLRTYLASVKA
MHEASKKLNECLQEVYEPDWPGRDEANKIAENNDLLWMDYHQKLVDQALLTMDTYLGQFPDIKSRIAKRGRKLV
DYDSARHHYESLQTAKKKDEAKIAKAEEELIKAQKVFEEMNVDLQEELPSLWNSRVGFYVNTFQSIAGLEENFHKEM
SKLNQNLNDVLVGLEKQHGSNTFTVKAQPSDNAPAKGNKSPSPPDGSPAATPEIRVNHEPEPAGGATPGATLPKSP
SQSSLPAVVVETFPATVNGTVEGGSGAGRLDLPPGFMFKVQAQHDYTATDTDELQLKAGDVVLVIPFQNPEEQDE
GWLMGVKESDWNQHKELEKCRGVFPENFTERVP
[Primer BIN1]
SEQ ID NO: 33
ACGGCGGGAAAGATCGCCAG
[Primer BIN1]
SEQ ID NO: 34
TTGTGCTGGTTCCAGTCGCT

The following examples are given for purposes of illustration and not by way of limitation.

### EXAMPLES

Abbreviations:

Aa or AA: amino acids; AAV: adeno-associated virus; DMSO: Dimethyl sulfoxide; EDTA: Ethylenediaminetetraacetic acid; HE: hematoxylin-eosin; KO: knockout; MTM: myotubularin; MTMR: myotubularin-related; PPIn: phosphoinositides; Ptdlns3P: phosphatidylinositol 3-phosphate; Ptdlns(3,5)P2: phosphatidylinositol 3,5-bisphosphate; SDH: succinate deshydrogenase; SDS: Sodium dodecyl sulfate; TA: tibialis anterior; Tg: transgenic; WT: wild type.

Materials and Methods

Materials

Primary antibodies used were rabbit anti-dysferlin (Abcam, AB15108, Cambridge, UK), anti-BIN1 (IGBMC), rabbit anti-DNM2 antibodies (IGBMC), and mouse β actin. Secondary antibodies against mouse and rabbit IgG, conjugated with horseradish peroxidase (HRP), were purchased from Jackson ImmunoResearch Laboratories (catalog 115-035-146 and 111-036-045). An ECL kit was purchased from Pierce.

Constructs used were pEGFP BIN1 (EGFP-tagged human BIN1 full length isoform 8: SEQ ID NO:29 and 30), pEGFP BIN1 ΔSH3 pAAV BIN1 (EGFP-tagged human BIN isoform 8, without exon 17: SEQ ID NO:27 and 28), pMyc DNM2 WT (myc-tagged human full length DNM2 wild-type cDNA), pMyc DNM2 R465W (myc-tagged human full length DNM2 cDNA with the R465W mutation), as well as the plasmids pGEX6P1 and pVL1392.

Recombinant proteins used were human BIN1 (whole) and SH3 of BIN1, human DNM2-12b (without exon 12b, corresponding to the main DNM2 isoform expressed in embryonic skeletal muscle; this isoform is also expressed in adult skeletal muscle) and DNM2+12b (with exon 12b, corresponding to the main DNM2 isoform expressed in adult skeletal muscle).

Proteins Purification

The pGEX6P1 plasmids encoding human BIN1 whole and SH3 of BIN1 proteins with GST tags (GST-BIN1 and GST-SH3) were produced from pGEX6P1 plasmid in E. coli BL21. E. coli producing these recombinant proteins were induced with IPTG (1 mM) for 3 hours at 37° C., centrifuged at 7,500 g, and then proteins were purified using Glutathione Sepharose 4B beads (GSH-resin).

Human DNM2-12b and DNM2+12b proteins were produced from pVL1392 plasmids encoding the dynamin genes in Sf9 cells with the baculovirus system. Briefly, a transfection was performed with DNM2 (±12b) plasmids to produce viruses. Sf9 cells were infected with viruses and grown for 3 days at 27° C., and then centrifuged at 2,000 g for 10 minutes. DNM2 recombinant proteins were purified with SH3 of BIN1 bound to Glutathione-Sepharose 4B beads (GE Healthcare).

The proteins after elutions were analyzed by 12% SDS-PAGE.

For the binding assays of DNM2 with BIN1, pure GST-BIN1 and GST-SH3 were loaded onto Glutathione Sepharose 4B beads, washed and incubated for 1 h at +4° C. with buffer without or with purified DNM2 -12b and DNM2+12b. After washing, the resin was analyzed by 12% SDS-PAGE.

Negative Staining

5 μl of DNM2 (90 ng. μL-1) and DNM2_BIN1 complex3 (150 ng. μL-1-1) were deposited onto 300 meshs Cu/Rh grids covered with a carbon film (Euromedex CF300-CU-050) freshly plasma cleaned (Fischione 1070). After 60 s of absorption, each sample was stained with 2% uranyl acetate and observed by electron microscopy with a FEI Tecnai F20 microscope operating at a voltage of 200 kV equipped with a Gatan US1000 detector. Images were recorded using the SerialEM software at a nominal magnification of 50 000×, yielding a pixel size of 2.12.

Liposomes Experiments

Liposomes were prepared mixing 5% PI(4,5)P2 (P-4516,Echelon Biosciences), 45% Brain Polar Lipids (141101C, MERK) and 50% PS (840035P, MERK) in a glass vial previously washed with chloroform. Then chlorofom was evaporated using nitrogen gas flow and 2 hr in a vacuum desiccator to create a transparent lipid film. The dried lipids were re-hydrated using the GTPase Buffer (20 mM HEPEs, 100 mM NaCl, 1 mM MgCl2, pH 7.4) to a final concentration of 1 mg/ml and went through three cycles of freezing (−80° C.) and defreezing (37° C.) each 15 minutes maintaining the vial in dark. The resulted liposomes were passed through 0.4 μm polycarbonate filters respectively 11 times using pre-hit Avanti Mini Extruder. The liposomes were stored in dark at 4° C. for max 24 h.

Liposomes were diluted to 0.17 mg/ml in GTPase Buffer and incubated with BIN1 and DNM2 as previously described by Takeda et al., 201828. BIN1, DNM2 or BIN1-DNM2 was diluted to 2.3 μM in the GTPase buffer. 10 μl of liposome solution were prepared on Parafilm and absorbed on EM carbon-coated grids for 5 minutes at room temperature in a dark humid chamber. The EM grids were transferred on droplets of BIN1, DNM2 or BIN1-DNM2 and incubated for 30 minutes at room temperature in dark. Then, the grids were incubated with 1 mM GTP for 5 minutes. Filter papaer was used to remove the solution. The EM grids were negatively stained as described in the previous paragraph.

In Cellulo Tubulation Assays

COS-1 cells plated in ibidi plate and grew in DMEM+1 g/L GLUCOSE+5% FCS to 70% confluence. Cells were transiently co-transfected with 0.5 uM BIN1-GFP plasmid and 0.5 uM or 1 uM DNM2-Myc or DNM2 RW-Myc using lifofectamin 3000 mix (L3000-015 Thermofisher) reagents in accordance with the manufacturer's protocol. After 24 hr of transfection, COS-1 cells were washed with phosphate-buffered saline (PBS) and fixed in 4% PFA diluted in PBS for 20 minutes. The cells were permeabilized with 0.2% of Triton X-100 diluted in PBS and after washing were blocked with 5% bovin serum albumin (BSA) in PBS for 1hr. COS-1 cells were incubated with primary antibody anti-DNM2 diluted in 1% BSA over-night. The secondary antibody anti rabbit Alexa 594 were diluted 1:500 and incubated for 2hr. COS-1 cells were observed on confocal microscope and only the co-transfected cells were considered. Cells with tubules considered shorter than tubules diameter were considered fragmented.

Mouse Lines

Mtm1-/y mouse line (129PAS) was previously generated and characterized (Buj-Bello, Laugel et al. 2002, Tasfaout, Buono et al. 2017). Mtm1 heterozygous females were obtained by homologous recombination of a target sequence, they were crossed with WT male to generate Mtm1-/y mice.

TgBIN1 (B6J) mice were obtained by the insertion of human BAC (n° RP11-437K23 Grch37 Chr2: 127761089-127941604) encompassing the full BIN1 gene with 180.52 Kb of genomic sequence. To obtain Dnm2^{RW/+} TgBIN1 mice, female Dnm2^{RW/+} was crossed with Tg BIN1 male.

The heterozygous Dnm2R465W/+ mouse line (C57BL/6J) was generated with an insertion of a point mutation in exon 11.

The homozygous Dnm2^{Rw/RW} TgBIN1 mice were generated by genetic cross of Tg BIN1 male and Dnm2R465W/+ female mice. The Dnm2R465W/+ Tg BIN1 mice were generated by crossing the Tg BIN1 with Dnm2R465W/+ whereas the Dnm2R465W/ R465W Tg BIN1 mice by crossing Dnm2R465W/+ Tg BIN1 male and Dnm2R465W/+ female.

Animals were maintained at room temperature with 12 hours light/ 12 hours dark cycle. Animals were sacrificed by cervical dislocation following European legislation on animal experimentation and experiments approved by ethical committees (APAFIS#5640-2016061019332648; 2016031110589922; Com'Eth 01594).

Animal Phenotyping, Hanging and Rotarod Tests

The phenotyping experiments were conducted blinded and all the experiments were repeated three time for each mouse, and by the same examiners, to ensure reproducibility and avoid stress. The daily phenotyping experiments were always performed in the same part of the day for all the mice in the cohort, while the weekly experiments were always performed on the same day of the week

The Hanging test was performed each week from 3 weeks to 8 weeks of age for the mouse line Dnm2^{RW/RW} TgBIN1 and every month from 1 to 7 month for Dnm2^{RW/+} TgBIN1 line. Mice were suspended from a cage lid for maximum 60 seconds and the test was repeated three times for each mouse at each time-point. The average time each mouse hang on the grid is presented in a graph.

The rotarod test was conducted at 4 and 8 months of age. The mice performed the test for 5 days long. During day 1 (“training day”), the mice were trained to run in acceleration mode on the rotarod. From day 2 to day5, mice were placed on the rotarod 3 times each day and they ran for a maximum of 5 minutes with increasing speed (4-40 rpm). Each mouse performed three times the test for each day in each time points. The data reported in the graph corresponded to the amount of time the animal run on the rotarod.

Muscle Force Measurement (TA Muscle Contraction)

Mice were anesthetized using Domitor (1 mg/kg), Fentanil (0.14 mg/kg) and Diazepam (4 mg/kg) by intraperitoneal injection. The sciatic nerve was detached and tied to an isometric transducer

The muscle force measurement on the tibialis anterior (TA) was then performed using a force transducer (Aurora Scientific) as described previously (Tasfaout, Buono et al. 2017). The absolute maximal force of the TA was measured after tetanic stimulation of the sciatic nerve with a pulse frequency from 1 to 125 Hz. The specific maximal force was determined dividing the absolute maximal force with the TA weight. After the measurement, mice were sacrificed by cervical dislocation and the TA muscle was extracted and frozen in liquid nitrogen-cooled isopentane and stored at −80° C.

AAV Transduction of Tibialis Anterior (TA) Muscle

The intramuscular injection was performed at 3 weeks old male wild-type, Mtm1-/y or Dnm2R465W/+ mice. The mice were anesthetized by intraperitoneal injection of ketamine (20 mg/ml) and xylazine (0.4%; 5 μl/g of body weight). The TA muscle was injected with 20 μl of AAV9 (7×10\hat{ }11 vg/mL) CMV human BIN1 construct (isoform 8 without exon 17), or with an empty AAV9 control diluted in physiological solution (PBS). The virus was produced by the molecular biology facility of the IGBMC. Animals post-injection were immediately housed in the ventilated cage.

Tissue Collection

Cervical dislocation was used to sacrifice mice after carbon dioxide suffocation. TA muscle was extracted and then frozen in isopentane cooled in liquid nitrogen. The muscles were stored at −80° C.

Histology

Transversal TA muscles cryosections of 8 μm were fixed and stained with Haematoxylin and Eosin (HE), nicotinamide adenine dinucleotide (NADH-TR) and succinate dehydrogenase (SDH) for histological analysis. After staining, images were acquired with the Hamamatsu Nano Zoomer 2HT slide scanner. Fiber size was measured by hand using Fiji software and fibers with abnormal SDH staining and nuclei position were counted using Cell Counter Plugin in Fiji software.

Tissue Immunolabeling

Transversal 8 μm cryosection slides were prepared from TA frozen in isopentane and stored at −80° C. After defreezing, and 3 PBS washes, the sections were permealized with 0.5% PBS-Triton X-100 and saturated with 5% bovine serum albumin (BSA) in PBS. The primary antibody dysferlin was diluted in 1% BSA and the secondary antibody was anti-rabbit and Alexa Fluor 488 were diluted 1:250 in 1% BSA.

Tissue Electron Microscopy

After dissection, TA was stored in 2.5% paraformaldehyde and 2.5% glutaraldehyde in 0.1M cacodylate buffer. Sections were observed by electron microscopy. To observe T-tubules, potassium ferrocyanide was added to the buffer (K3Fe(CN) 6 0.8% , Osmium 2%, cacodylate 0.1M)(Al-Qusairi, Weiss et al. 2009). The triad number per sarcomere and T-tubule direction were measured manually using Fiji program.

Protein Extraction and Western-Blot

TA muscle was lysed in RIPA buffer with 1 mM DMSO, 1mM PMSF and mini EDTA free protease inhibitor cocktail tablets (Roche Diagnostic) on ice. The protein concentration was measured using the BIO-RAD Protein Assay Kit (BIO-RAD). Loading buffer (50 mM Tris-HCl, 2% SDS, 10% glycerol) was added to protein lysates, and proteins were separated by 8% or 10% in SDS-polyacrylamide gel electrophoresis containing 2,2,2-Trichloroethanol (TCE) in order to visualize all tryptophan-containing proteins. After transfer to nitrocellulose, saturation was done with 3% BSA or 5% milk, primary antibody and secondary antibody was added: β1 integrin (MAB1997, 1:500), vinculin (V9131, 1:1000), BIN1 (1:1000; IGBMC), MTM1 (2827, 1:1000; IGBMC), GAPDH (MAB374, 1:100000).

Statistical Analysis

All the data are expressed as mean±s.e.m. GraphPad Prism software versions 5&6 was used to generate the graphs and the statistic tests. The unpaired students T-test was used to compare two groups when they followed a normal distribution. To compare more than two groups which followed a normal distribution, one-way ANOVA and Tukey's post hoc test were used. If the groups did not follow a normal distribution, no parametric Kruskal Wallis test and Dunn's post-hoc were applied. P values smaller than 0.05 were considered significant. The number of mice and the tests used for each experiment are indicated in the figure legends.

Results

Generation of Dnm2^{R465W/+} Tg BIN1 mouse line

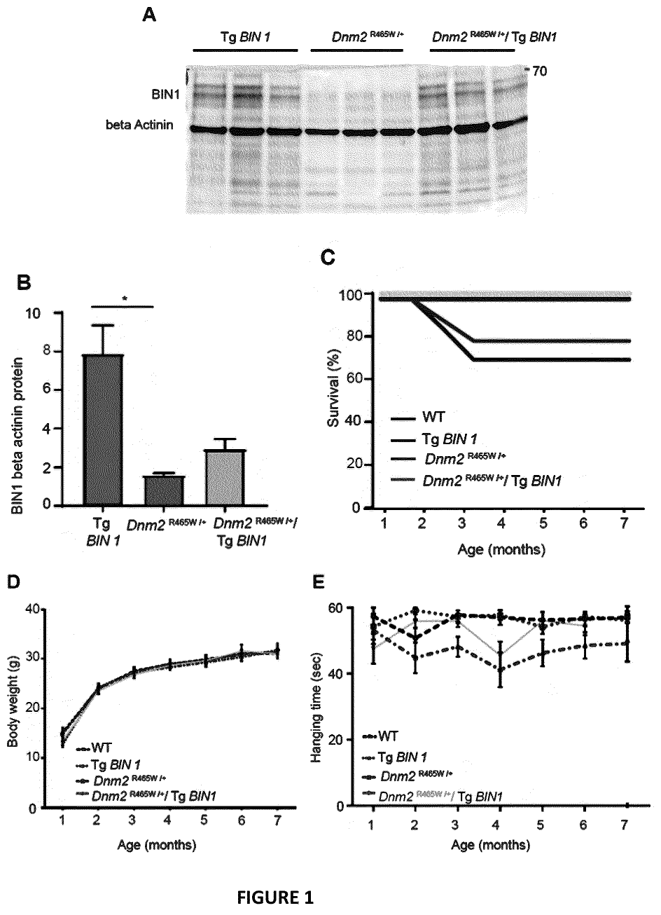

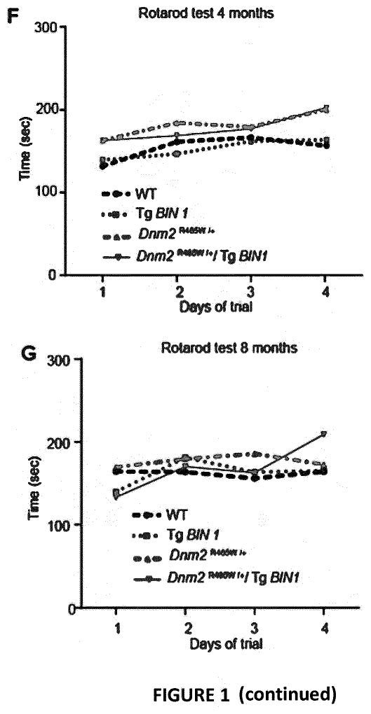

To study the effect of BIN1 overexpression on a DNM2-CNM mutation in vivo, female Dnm2^{R465W/+} mice (Durieux et al., 2010) were crossed with Tg BIN1 mice expressing human BIN1 from a bacteria artificial chromosome to produce Dnm2^{R465W/+} Tg BIN1 mice. No differences were observed in BIN1 protein level between the Tibialis Anterior (TA) lysate of WT and the Dnm2^{R465W/+} mice (data not shown). An increase of 8-fold and 3-fold was detected in Tg BIN1 mice and in Dnm2^{R465w/+} Tg BIN1 compared to Dnm2^{R465W/+} respectively (FIG. 1A-B).

Most of the mice analyzed survived until the end fixed of the study (7 months of age), and only some WT (28.5%) and Dnm2^{R465W/+} (18%) died for unknown problems (FIG. 1C). No difference was identified in body weight between WT, TgBIN1, Dnm2^{R465W/+} and Dnm2^{R465W/+} TgBIN1 mice throughout the 7 months analyzed in this study (FIG. 1D).

Characterization of Dnm2^{R465W/+} Tg BIN1 Mouse Model Phenotypes

Previous results showed that Dnm2^{R465W/+} have normal growth (Durieux et al., 2010).

To verify if the increased BIN1 expression ameliorated the reduced skeletal muscle force reported in the Dnm2^{RW/+}, hanging and rotarod test were performed at different time points. Dnm2^{R465W/+} hang on the grid slightly less than the Dnm2^{R465W/+} TgBIN1 and the control genotypes (TgBIN1 and the WT mice) (FIG. 1E).

To assess if the Dnm2^{R465W/+} exhibited a problem in general coordination, the rotarod test was performed at 4- and 8-month mice using different mice cohort. Mice were placed on the rotarod for 5 minutes in acceleration mode and the test was repeated for 4 days for each cohort. No difference in time spent on the rotarod have been identified between all the mice genotypes; the Dnm2^{R465W/+} performed better than the WT and TgBIN1 control mice (FIG. 1F-G).

Overall, these results suggest that the overexpression of BIN1 positively impacted on the total body muscle force of Dnm2^{RW/+} mice.

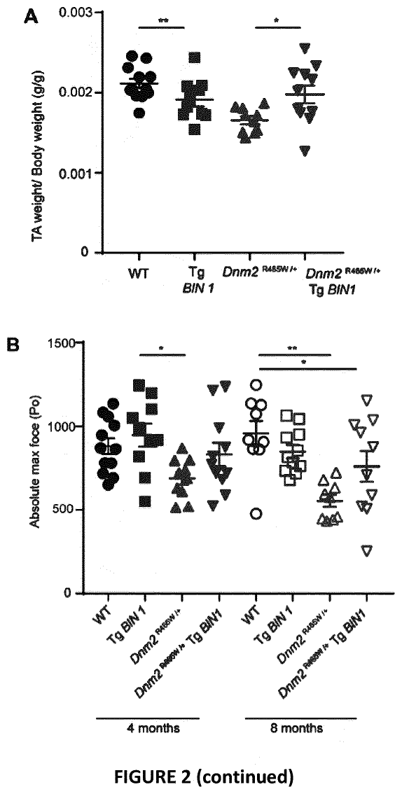

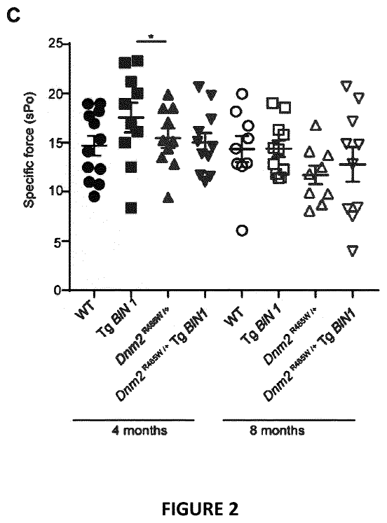

We then verified if the force of the TA muscle was impaired. Previous publications showed atrophy in Dnm2^{R465W/+} TA muscle from the second months of age (Durieux et al., 2010) (Buono et al., 2018). We analysed the TA muscle at 4 months of age, the overexpression of BIN1 significantly rescued the TA muscle weight of Dnm2^{R465W/+} mice (FIG. 2A). We then tested the absolute TA muscle force. The absolute TA muscle force was significantly reduced in Dnm2^{R465W/+} mice compared to the TgBIN1 and WT control mice at 4- and to the WT at 8-month of age (FIG. 2B). The overexpression of BIN1 in Dnm2^{R465W/+} ameliorated the absolute muscle force at 4- and 8-month (FIG. 2B). Next, the specific in situ TA muscle force was measured: no significant difference was identified at 4-month of age between the Dnm^{R465W/+} mice and the control phenotypes suggesting that this time-point the phenotype of the mice is still not severe. A trend of improvement was observed in the Dnm2^{R465W/+} Tg BIN1 at 8-month compared to the Dnm2^{R465W/+} mice (FIG. 2C).

To conclude, Dnm2^{R465W/+} mice exhibited a slight defect in total body strength and no difference in coordination and motor activity with the WT control. However, the overexpression of BIN1 rescued TA muscle weight and slightly improved absolute muscle force at 4 and 8-month of age: indeed, Dnm2^{RW/+} mice exhibited a slight improvement in total body strength and a complete rescue of the muscle atrophy compared to the Dnm2^{RW/+} disease model.

Overexpression of BIN1 Level Rescues the Histological Features in Dnm2^{R465W/+} Muscles: BIN1 Improves CNM Histological Features

To verify if the improvement in TA muscle weight and muscle force observed in Dnm2^{R465W/+} TgBIN1 mice correlates with an improvement in Dnm2^{R465W/+} muscle structure, we analyzed the TA muscle histology and ultrastructure features. To do so, transversal TA sections were stained with hematoxylin and eosin (HE).

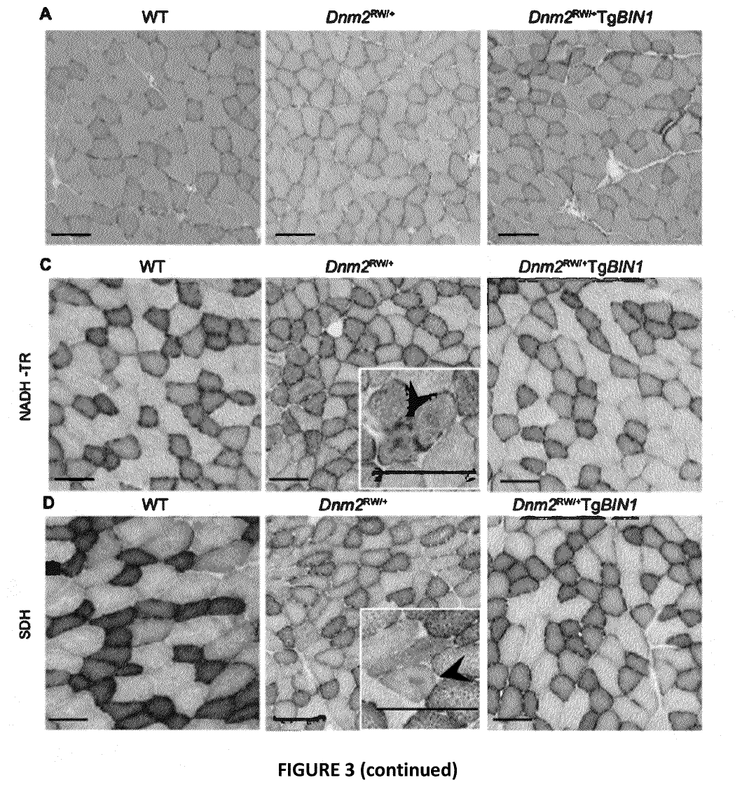

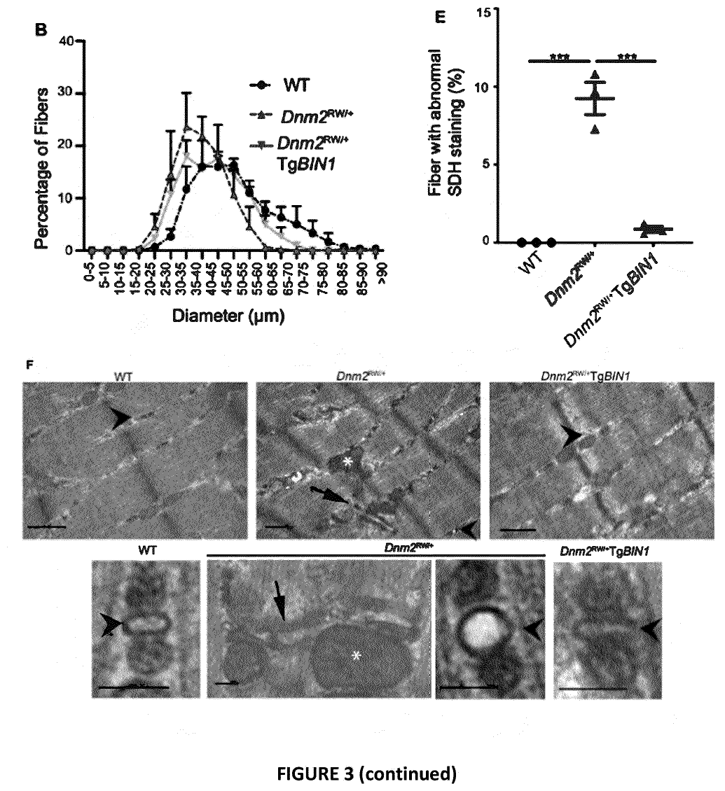

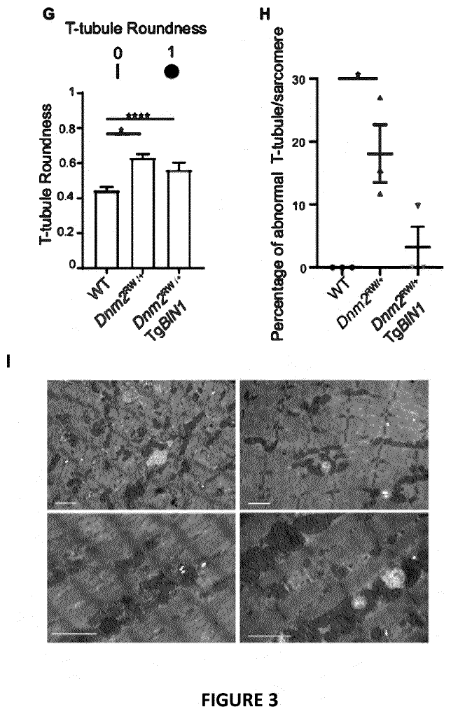

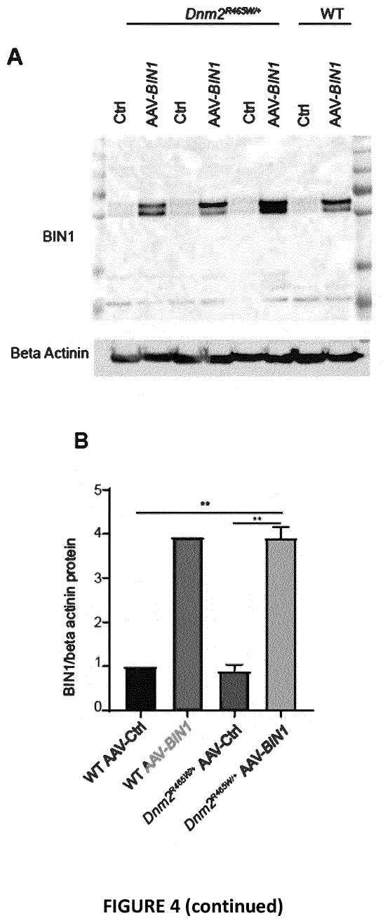

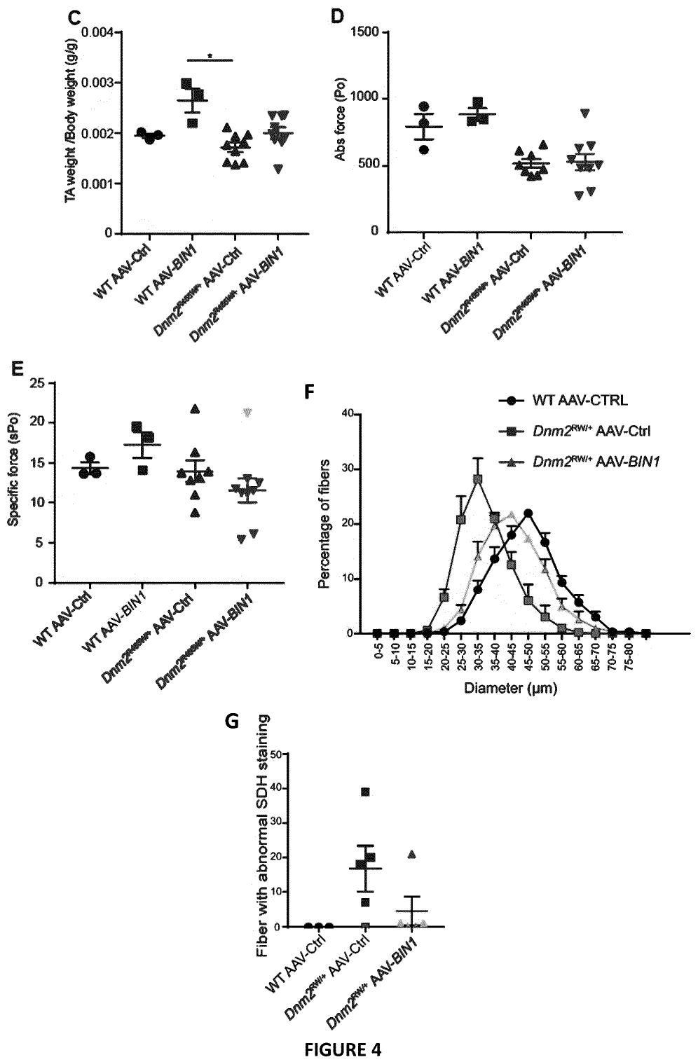

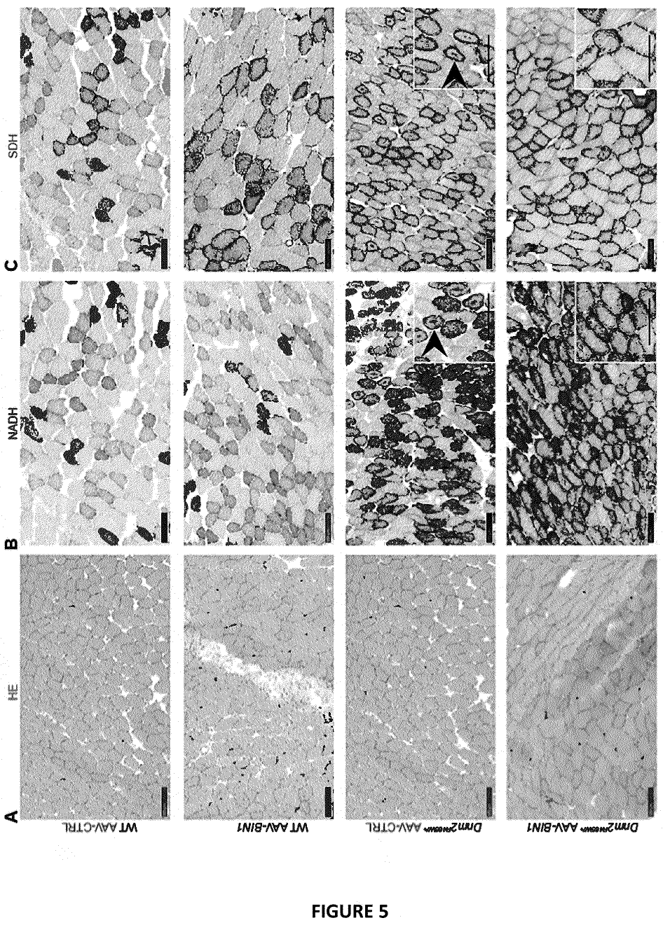

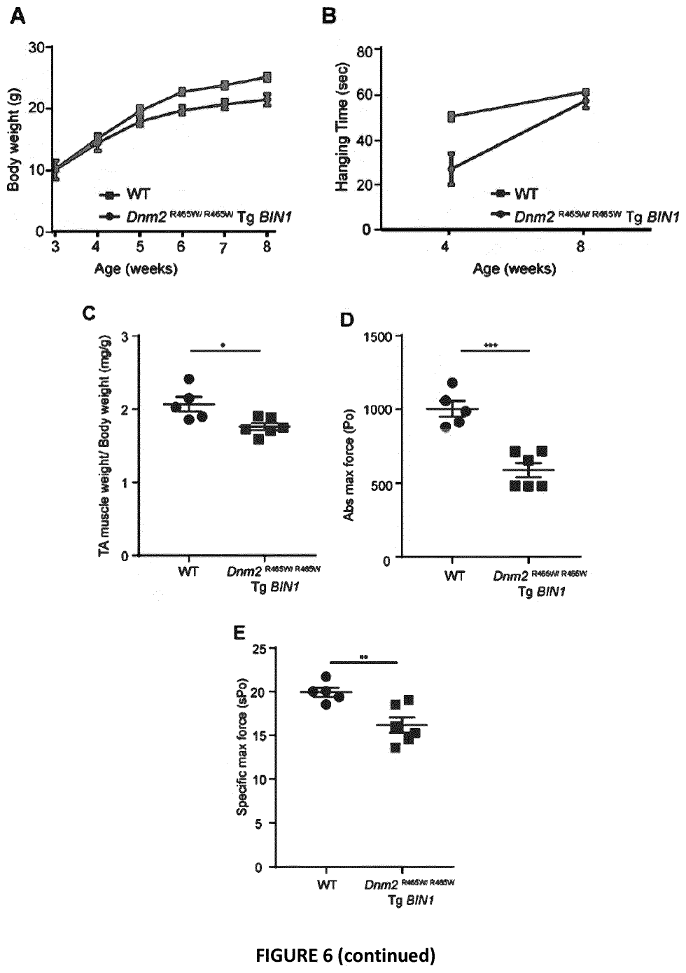

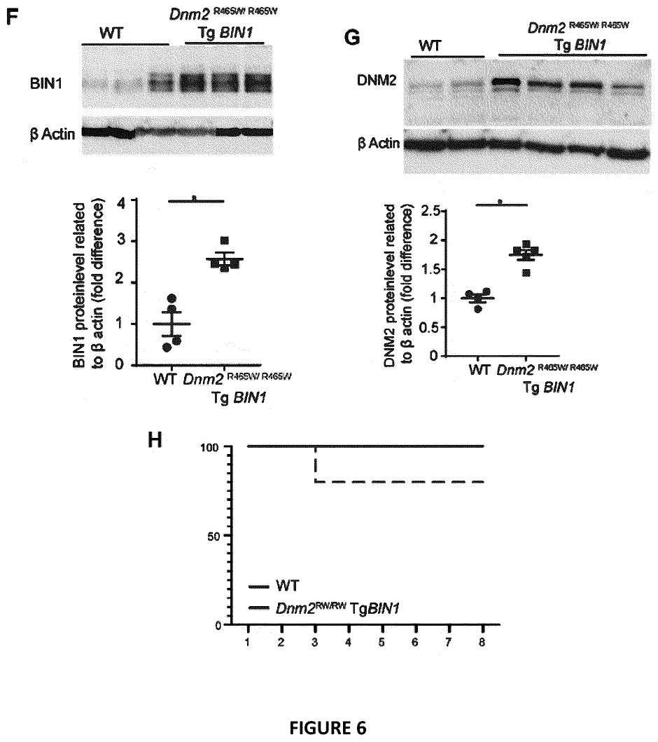

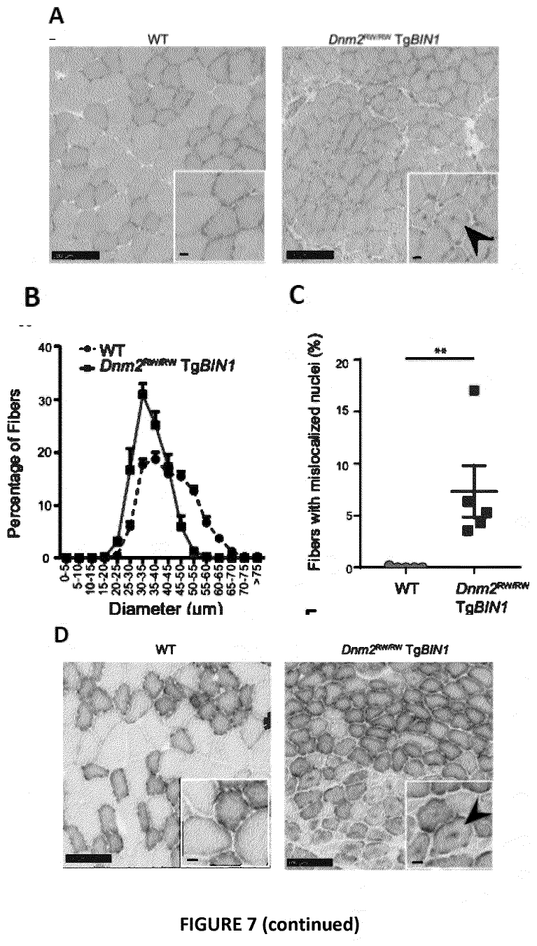

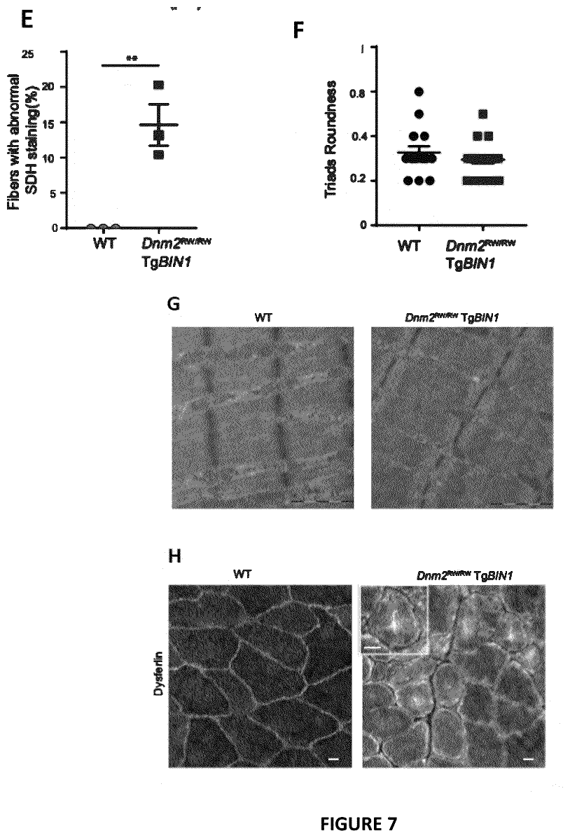

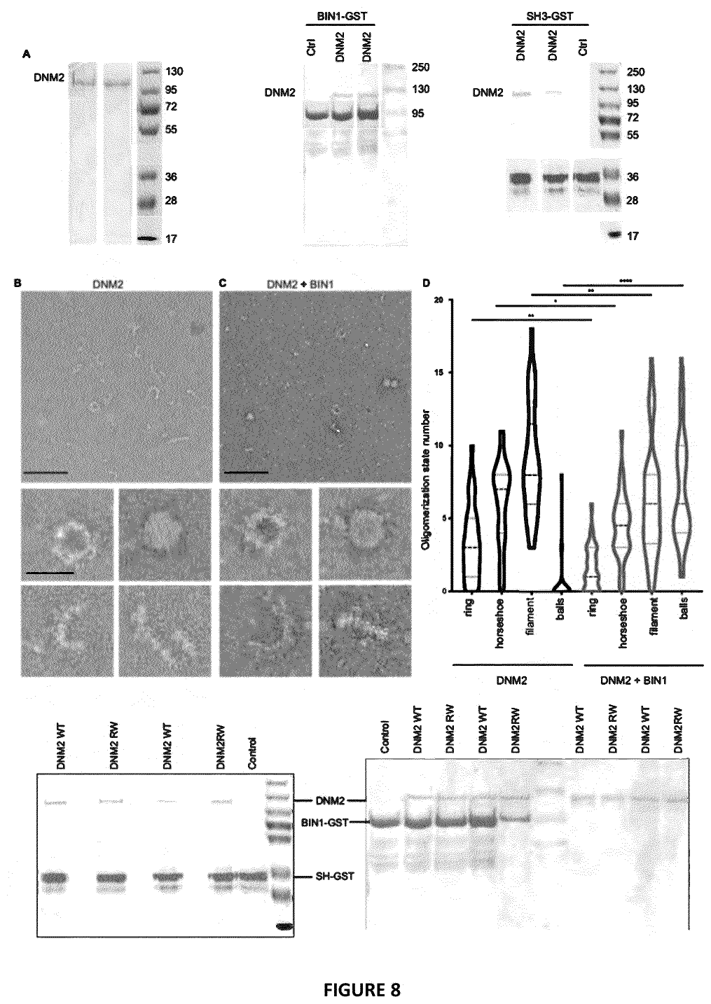

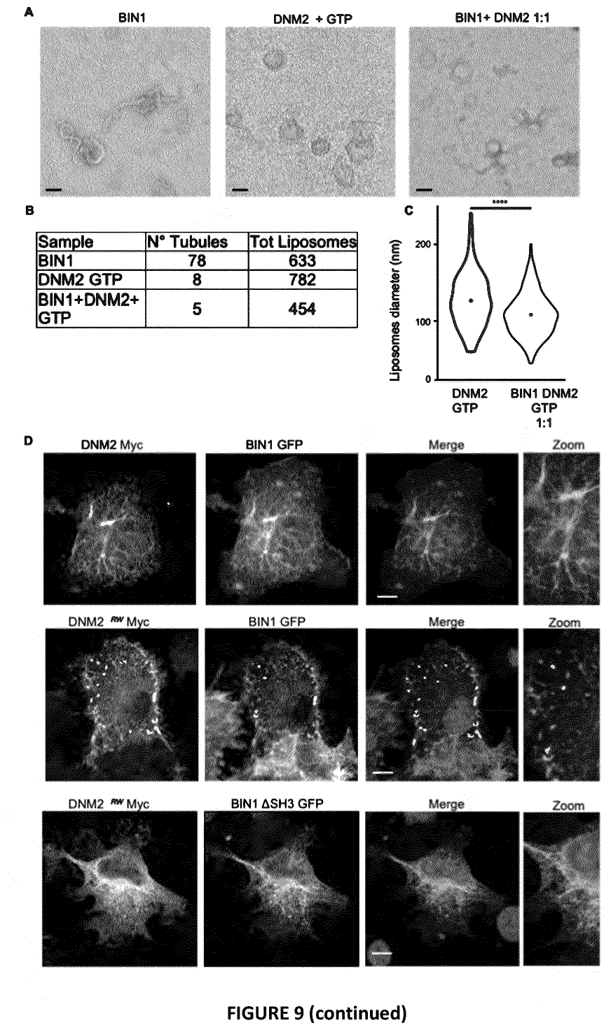

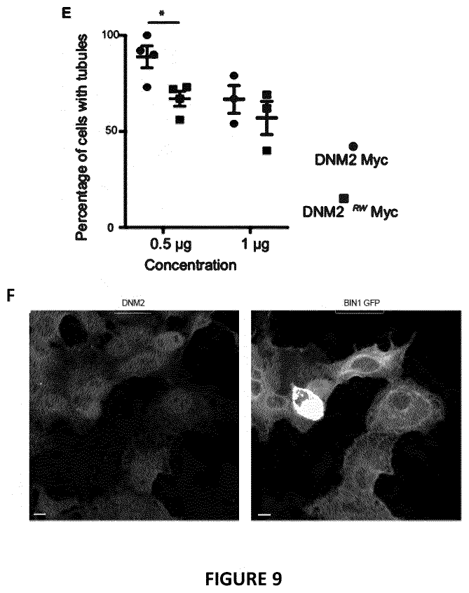

At 4 months, no difference in nuclei position and fiber size was identified between Dnm2^{RW/+} and Dnm2^{R465W/+} TgBIN1 and controls (FIG. 3 A-B). The main histological feature of Dnm2^{RW/+} mice was the abnormal aggregation of NADH-TR and SDH staining in the middle of the muscle fibers (Durieux et al., 2010). This finding was confirmed upon succinate dehydrogenase (SDH) and nicotinamide adenine dinucleotide (NADH-TR) stainings: indeed, this abnormal staining was detectable at 4 m and 8 m of age in Dnm2^{R465W/+} l TA (FIG. 3C, arrows and FIG. 3D). The overexpression of BIN1 in Dnm2^{R465w/+} mice restored the control (WT) phenotype (Tg BIN1) at 4 months (FIG. 3E). SDH staining specifically labels mitochondria activity. Therefore, overexpression of BIN1 by genetic cross improves the histological defects observed in Dnm2^{R465W/+} mice.

Skeletal muscle ultrastructure was investigated by electron microscopy. Dnm2^{RW/+} muscle presented enlarged mitochondria that were often found clustered, correlating with the accumulation of oxidative staining (FIG. 3I). T-tubules transversal section was rounder in Dnm2^{RW/+} and Dnm2^{RW/+} TgBIN1 mice compared to WT (FIG. 3F-K). We excluded that this phenotype was due to the overexpression of BIN1 as previous analysis did not identify abnormalities in the TgBIN1 . However, T-tubule orientation was altered and more longitudinal in Dnm2^{RW/+} mice and rescued in Dnm2^{RW/+} TgBIN1 mice (FIG. 3H). Overall, the overexpression of BIN1 rescued the abnormal mitochondria organization representing the main histopathological feature in common between the Dnm2^{RW/+} mice and DNM2-CNM patients.

The Post-Natal Overexpression of BIN1 Improves Dnm2^{RW/+} Muscle Atrophy and Histological Muscle Features

Dnm2^{R465W/+} Tg BIN1 mice were obtained by genetic cross and BIN1 was overexpressed since in utero. To develop a translated therapeutic approach, we aimed to modulate BIN1 expression after birth. To do so, human BIN1 isoform 8 (without exon 17, i.e. corresponding to SEQ ID: 27 and 28), which is the main BIN1 isoform expressed in adult skeletal muscle in mice and human, was overexpressed using adeno-associated virus (AAV) delivery: in short, AAV-BIN1 was injected intramuscularly in 3-week old Dnm2^{R465W/+} mice that were subsequently analyzed 4 weeks post-injection. A 4-fold of increase in BIN1 expression was detected in the muscles of Dnm2^{R465W/+} mice injected with AAV-BIN1 compared to the contralateral leg injected with AAV-Ctrl (FIG. 4 A-B). The increase of BIN1 expression allowed a slight improvement of TA muscle weight in Dnm2^{R465W/+} leg injected with AAV-BIN1 compared to the leg injected with AAV-Ctrl (FIG. 4 C). The WT TA injected with AAV-BIN1 weighted more than the control leg (FIG. 4 C). No improvement in absolute and specific muscle force was detected in the Dnm2^{RW/+} TA muscles injected with either AAV-BIN1 or AAV-Ctrl (FIG. 4 D-E).

At 7 weeks, reduction in fiber size was noted in the Dnm2^{RW/+} injected with AAV-Ctrl, as found at the same age in Dnm2^{RW/+}. This was partially rescued with AAV-BIN1 (FIG. 5A and 4F). The injection of AAV-BIN1 ameliorated the main Dnm2^{RW/+} histological defect. The central accumulation of NADH-TR and SDH stainings observed in Dnm2^{RW/+} TA injected with AAV-Ctrl were not visible upon injection with AAV-BIN1 (FIG. 5 and 4G).

In summary, the exogenous expression of human BIN1 in Dnm2^{R465W/+} TA muscle, via AAV, improved the central accumulation of oxidative activity but not the muscle force after 4 weeks of expression. Muscle force was however improved via genetic crossing. An improvement in muscle force would most likely be observed via AAV-BIN1, should the viral vector be administered a bit earlier and/or mice receiving AAV-BIN1 had been analyzed at a later time point.

Overexpression of BIN1 Prevents the Premature Lethality of Dnm2^{R465W/R465W} Mice

Since the overexpression of BIN1 in utero was able to improve the Dnm2^{R465W/+} muscle atrophy/weight and histopathology, we next tested if the overexpression of BIN1 rescues the life span of homozygous Dnm2^{R465W/R465W} mice, which model the most severe phenotype of ADCNM. The Dnm2^{R465W/R465W} mice were previously described to survive for a maximum of 2 weeks postnatally, and surviving mice presented severe muscle phenotypes (Durieux et al., 2010).

To do so, Dnm2^{R465W/R465W} mice overexpressing BIN1 in utero were generated and female Dnm2^{R465W/+} were then crossed with male Dnm2^{R465W/+} Tg BIN1 mice. At 10 d, only 0.7% of the pups analyzed were Dnm2^{RW/RW} mice suggesting that the majority died before, while 18% were Dnm2^{RW/RW} TgBIN1 corresponding to the expected Mendelian ratio (Table 1) and all the mice survived until 8 weeks (FIG. 6H). A small cohort of Dnm2^{RW/RW} TgBIN1 mice were followed-up and strikingly survived until 18 months, the normal lifespan for WT mice.

TABLE 1
Percentage of male pups genotypes at 10 days post-birth during the generation
of Dnm2^{RW/RW} TgBIN1 mice (total mice analyzed = 138).
Female Dnm2^{R465W/+} × Male Dnm2^{R465W/+} Tg BIN1

| | | | | | Dnm2^{RW/+} | Dnm2^{RW/RW} |
|---|---|---|---|---|---|---|
| Only Male | WT | Dnm2^{RW/+} | Dnm2^{RW/RW} | TgBIN1 | TgBIN1 | TgBIN1 |
| Expected | 16.7% | 16.7% | 16.7% | 16.7% | 16.7% | 16.7% |
| Obtained | 24.6% | 26.8% | 0.7% | 18.8% | 10.9% | 18.1% |
| at PN 10 d | | | | | | |

The overexpression of BIN1 was confirmed by Western Blot (FIG. 6F): a 2-fold overexpression of BIN1 was sufficient to rescue the life span of the Dnm2^{R465W/R465W} mice. Only a slight difference was observed in Dnm2^{R465W/R465W} Tg BIN1 mice, which weighed less than the WT control from 6 weeks of age (FIG. 6A).

Overall, these results show that increasing BIN1 expression is sufficient to rescue neonatal lethality and lifespan of Dnm2^{R465W/R465W} mice.

### Characterization of Dnm2^{R465W R465W T}g BIN1 Mice Phenotype and Muscle Force

Since the overexpression of BIN1 rescued the Dnm2^{R465W/R465W} survival, we characterized their motor function and muscle phenotypes at 2 months. To do so, the total body force and specific in situ muscle force were measured.

To assess the total body strength, the hanging test was performed. At 4 weeks old Dnm2 R465W/ R465W Tg BIN1 were able to hang for up to 20 seconds to the grid. At 8 weeks of age, no difference was observed between the Dnm2^{R465W/R465W} Tg BIN1 and the WT control (FIG. 6 B).

We next analyzed the TA muscles: Dnm2^{R465W/R465W} Tg BIN1 had smaller TA muscles compared to the WT control (FIG. 6 C). A significant difference was obtained between the WT and Dnm2^{R465W/R465W Tg} BIN1 TA muscle absolute and specific force (FIG. 6 D-E). A significant difference of muscle absolute and specific force was noted between Dnm2^{RW/RW} TgBIN1 and WT mice (FIG. 6 E-F). Dnm2^{R465W/R465W} Tg BIN1 mice had a TA absolute force of 600 mN which was a similar value as for Dnm2^{R465/+} mice (FIG. 2 B). In addition, we verified the level of DNM2 on the TA lysates of Dnm2^{R465W/R465W} Tg BIN1 mice: it was significantly higher compared to WT (FIG. 6 G). To conclude, the Dnm2^{R465W/R465W} Tg BIN1 have normal body strength but lower TA muscle strength than the WT control at 8 weeks. In other words, while the muscle force was not at WT level, it was sufficient for a normal motor function measured in the hanging test.

Characterization of Dnm2^{R465W/R465W} Tg BIN1 Muscle Histology and Ultrastructure

To assess the skeletal muscle histology and structure, TA muscles were analyzed after histological staining with HE and showed reduced fiber diameter in Dnm2^{RW/RW} TgBIN1 mice compared to WT (FIG. 7G-H). In addition, HE transversal muscle sections staining (FIG. 7 A) showed a small percentage of fibers with nuclei abnormally positioned (around 7%) in Dnm2^{R465W/R465W} Tg BIN1 TA muscle (FIG. 7 C), while this CNM phenotype was not observed in Dnm2^{RW/+} mice (FIG. 3). In addition, abnormal internal dark staining was visible in some muscle fibers stained with HE and SDH (arrows) (FIG. 7 A-and D). Around 15% of Dnm2^{R465W/R465W} Tg BIN1 TA muscle fiber had abnormal SDH aggregates (i.e. abnormal central accumulation of oxidative activity) (FIG. 7 D-E). Fiber with abnormal aggregates were mainly situated on the periphery of the TA muscle.

Electron microscopy pictures did not reveal abnormalities in muscle ultrastructure in Dnm2^{RW/RW} TgBIN1 mice and showed aligned Z-lines and normal muscle triads localization and shape (FIG. 7 F-G), unlike the heterozygous Dnm2^{RW/+} mice (FIG. 3). Dysferlin, a protein involved in membrane repair and T-tubule biogenesis and usually present at the sarcolemma in adult muscle, was mainly accumulated inside myofibers (FIG. 7 H). As T-tubules have a normal shape and orientation by electron microscopy, dysferlin defects may underline the alteration of another membrane compartment. Of note, dysferlin intracellular accumulation in Dnm2^{RW/+} mice has been previously been reported in the literature.

In conclusion, Dnm2^{R465W/R465W} Tg BIN1 had defects in nuclei position and SDH staining compared the WT control. In others words, Dnm2^{RW/RW} TgBIN1 mice displayed most phenotypes found in the Dnm2^{RW/+} mice and reminiscent of CNM but otherwise their muscle ultrastructure was rather preserved.

BIN1 Affects DNM2 Oligomer Structure

The above data support that BIN1 is a modulator of DNM2 in vivo.

To better decipher their functional interaction at the molecular level, experiments in cells and in vitro were conducted. First, the interaction between human DNM2 with human BIN1 was tested by pulldown of recombinant DNM2 produced in insect cells with recombinant GST-BIN1 (full length isoform 8) or GST-BIN1-SH3 (SH3 alone) produced in bacteria. BIN1 interacted with DNM2 (FIG. 8 A and E). The oligomer structure of human DNM2 was assessed by negative staining and electron microscopy. DNM2 can assemble as filament, horseshoe or rings (FIG. 8 B). Addition of BIN1 biased the oligomer representation of DNM2 (typically in a form of filaments, horseshoe or ring) towards a fourth structure resembling a “ball”, while the ball structure was barely present with DNM2 alone (FIG. 8 C-D; arrow). These data suggest that BIN1 affects the oligomer structure of DNM2.

The BIN1-DNM2 Complex Regulates Membrane Tubulation

To investigate in more details the function regulated by the BIN1-DNM2 complex, we turned to membrane tubulation.

To do so, liposomes supplemented with phosphatidylserine and PtdIns(4,5)P₂ were incubated with BIN1, DNM2, or BIN1 and DNM2 and analyzed by negative staining. BIN1 generated membrane tubules from liposomes (78 tubules on 633 liposomes counted, 13% of tubulating liposomes) while nearly no tubules were noted with DNM2 with GTP (8 tubules on 782 liposomes counted, 1% of tubulating liposomes) (FIG. 9 A-B). Addition of DNM2 with GTP to BIN1 in a 1:1 ratio resulted in liposomes without tubules (5 tubules on 454 liposomes counted), suggesting DNM2 either prevented or cut the tubules made by BIN1 (FIG. 9 B). To distinguish between the two possibilities, the diameter of the resulting liposomes was measured and found to be reduced when BIN1 was added to DNM2 (FIG. 9 C). The mean liposome diameter was 126.66+/−2.8 for DNM2 alone and 108.283+/-1.89 DNM2 with BIN1.

Overall, these data support that BIN1 and DNM2 work together to promote membrane tubules fission.

The DNM2 R465W CNM Mutation Alters the Fission Property of DNM2 in Cells

To confirm that the BIN1-DNM2 complex regulates membrane tubulation in living cells, BIN1+/−DNM2 was overexpressed in COS-1 cells.

BIN1 expression induced intracellular membrane tubules mainly originating from the plasma membrane (FIG. 9F). Co-expressed DNM2 WT co-localized with BIN1 on tubules which number decreased upon cell transfection with a higher concentration of DNM2 DNA, confirming that BIN1 recruits DNM2 to fission the tubules as suggested by the liposome data (FIG. 9 D). In co-transfected cells without tubules, BIN1 and DNM2 co-localized to intracellular dots probably representing the product of tubules fission. Co-expression of BIN1 with DNM2 R465W CNM mutant at low concentration led to a lower number of cells with tubules compare to co-expression with DNM2 WT (FIG. 9 E). The SH3 domain of BIN1 was necessary to recruit DNM2 to the tubules as a BIN1 ΔSH3 protein lacking the SH3 domain was not able to recruit DNM2. In conclusion, BIN1 and DNM2 act together on membrane tubule fission and the DNM2-CNM mutation alters this process.

### DISCUSSION

In this study, we report that exogenous expression of human BIN1 ameliorates the muscle phenotype of Dm2^{RW/+} mice, the mammalian model for centronuclear myopathy linked to DNM2 mutations, and the perinatal lethality of homozygous Dnm2^{RW/RW} mice. These data demonstrate that increasing BIN1 can be used as a therapy for this form of centronuclear myopathy. In addition, in vitro and cell experiments supports that BIN1 directly binds to DNM2, is necessary for its recruitment to membrane tubules, and that the BIN1-DNM2 complex regulates tubules fission. Altogether, BIN1 appears to be an in vivo modulator of DNM2.

BIN1 is an In Vivo Modulator of DNM2

We demonstrated herein that BIN1 overexpression in the Dnm2^{RW/+} mice rescues the muscle phenotype. This mechanism is not fully understood, though it is conceivable that BIN1 and DNM2 act together on membrane tubule fission, by potentially binding to each other through their respective SH3 and PRD domains. Dynamin activity on membranes may then be regulated by the clustering of PIP2 induced by BIN1. In cells, DNM2 is recruited to BIN1 induced membrane tubules and increasing DNM2 promoted membrane fission (FIG. 8 E). Similarly, the addition of BIN1 to DNM2 on liposomes led to reduction in liposome size (FIG. 8 B-D).

The DNM2-CNM mutant R465W alters DNM2 fission activity in cells (FIG. 8 E). In addition, BIN1 can modulate specifically this mutant in vivo as overexpression of BIN1 rescued the lifespan of the homozygous Dnm2^{RW/RW} mice (FIG. 4). The R465W DNM2 mutation leads to an increased GTPase activity and membrane fission. Overall, BIN1 and DNM2 act together on membrane tubule fission and the DNM2-CNM mutation alters this process, in all likelihood through, a «gain-of-function» mechanism. BIN1 would induce membrane curvature, recruit DNM2 to these membrane sites and promote its fission activity that is increased by the DNM2-CNM mutation.

In cardiac and skeletal muscle, BIN1 was proposed to regulate T-tubule biogenesis. T-tubules are plasma membrane invagination crucial for intracellular calcium release and contraction. Alteration of T-tubule and triad orientation and shape was noted in the Dnm2^{RW/+} mice (FIG. 1), in WT mice transduced with AAV overexpressing the R465W DNM2-CNM mutant, and in drosophila and zebrafish overexpressing the same mutant. It is thus possible that the BIN1-DNM2 complex regulates T-tubule biogenesis or/and maintenance. It can however not be excluded that this complex also regulates other cellular functions, since BIN1 expression clearly rescued the central accumulation of mitochondria oxidative activity in myofibers, a key hallmark of CNM (FIGS. 1-4).

Increasing BIN1 as a Therapy to Counteract DNM2 Mutations

The present data show that it is possible to rescue the AD-CNM muscle phenotype via BIN1.

The “proof-of-concept” (POC) was provided herein by demonstrating that exogenous BIN1 expression in utero can rescue heterozygote DNM2-CNM mice, which model a mild form of ADCNM. This POC was then translated through AAV-BIN1 delivery post-birth.

The next experiments were then performed in mice mimicking a severe form of ADCNM (homozygote Dnm2^{RW/RW} mice): BIN1 overexpression also rescued the muscle phenotype/function and improved the lifespan of these mice. Interestingly, the Dnm2^{RW/RW} TgBIN1 mice exhibited muscle atrophy, a decrease muscle force and a central accumulation of nuclei and oxidative activity in myofibers which did not affect their survival. Noteworthy, these alterations are similar to those observed in untreated Dnm2^{RW/+} mice (no BIN1 expression), which suggest that BIN1 expression transforms a severe DNM2-CNM disease into a very mild disease form. The present data also show that BIN1 expression can improve both the childhood onset DNM2-CNM form mainly due to R465W mutations and the severe neonatal form mainly due to other missense mutation

The present data also investigates BIN1 and DNM2 functional relationship, and shows that it is crucial for skeletal muscle integrity.

Modulating BIN1 level, in particular the muscle-specific BIN 1 isoform, can thus represent a novel therapy for autosomal-dominant centronuclear myopathy.

### CONCLUSION

Overexpression of BIN1 can be used as an effective treatment of DNM2-CNM, whether as a severe or mild form, i.e. at early or late onset of the disease.

1-16. (canceled)

17. A method of treating autosomal-dominant centronuclear myopathy (ADCNM) comprising the administration of an Amphiphysin 2 polypeptide or a BIN1 nucleic acid sequence to a subject in need of treatment.

18. The method according to claim 17, wherein the BIN1 nucleic acid sequence comprises the sequence represented by SEQ ID NO: 1 or comprises a sequence comprising any combination of at least two or three different BIN1 exons 1-20 represented by SEQ ID NO: 3-22, respectively.

19. The method according to claim 18, wherein the BIN1 nucleic acid sequence comprises any combination of at least two or three different BIN1 exons 1-20 represented by SEQ ID NO: 3-22, respectively, and according to increasing numbering of exons 1-20.

20. The method according to claim 17, wherein the BIN1 nucleic acid sequence is a nucleic acid sequence comprising at least exons 1 to 6 and 8 to 11, a nucleic acid sequence represented by SEQ ID NO: 23, a nucleic acid comprising at least exons 1 to 6, 8 to 10, 12, and 17 to 20, a nucleic acid sequence represented by SEQ ID NO: 25, a nucleic acid comprising at least exons 1 to 6, 8 to 10, 12, and 18 to 20, a nucleic acid sequence represented by SEQ ID NO: 31, a nucleic acid sequence comprising at least exons 1 to 6, 8 to 12, and 18 to 20, a nucleic acid sequence represented by SEQ ID NO: 27, a nucleic acid sequence comprising at least exons 1 to 6, 8 to 12, and 17 to 20, a nucleic acid sequence represented by SEQ ID NO: 29, or the BIN1 nucleic acid sequence that hybridizes or is complementary to the sequence of SEQ ID NO:1, 23, 25, 27, 29 or 31.

21. The method according to claim 17, wherein the amphiphysin 2 polypeptide comprises a polypeptide sequence represented by SEQ ID NO: 2 or any polypeptide sequence deriving therefrom or encoded by any combination of at least two different BIN1 exons 1-20, represented by SEQ ID NOs: 3-22, respectively.

22. The method according to claim 17, wherein the amphiphysin 2 polypeptide comprises a polypeptide sequence deriving therefrom or encoded by any combination of at least two different BIN1 exons 1-20, represented by SEQ ID NOs: 3-22, respectively, and according to increasing numbering of exons 1-20.

23. The method according to claim 21, wherein the amphiphysin 2 polypeptide comprises an amino acid sequence represented by SEQ ID NO: 2, 24, 26, 28, 30 or 32, or an amino acid sequence at least 90% identical to SEQ ID NO: 2, 24, 26, 28, 30 or 32, or a bioactive fragment or variant thereof.

24. The method according to claim 17, wherein the amphiphysin 2 polypeptide comprises an amino acid sequence that is at least 80% identical to the naturally occurring Amphiphysin 2 of SEQ ID NO: 2, 26, 28, 30 or 32.

25. The method according to claim 17, wherein the BIN1 nucleic acid sequence is operably linked to one or more control sequences that direct the production of Amphiphysin 2 polypeptide.

26. The method according to claim 17, wherein the BIN1 nucleic acid sequence is in a recombinant expression vector.

27. The method according to claim 26, wherein the recombinant expression vector is an expression viral vector.

28. The method according to claim 27, wherein the viral vector is an adeno-associated viral (AAV) vector or an AAV9 vector.

29. The method according to claim 26, wherein the recombinant expression vector is comprised in a recombinant host cell.

30. The method according to claim 17, wherein the Amphiphysin 2 polypeptide, BIN1 nucleic acid sequence, recombinant expression vector, or recombinant host cell is comprised in a pharmaceutical composition.

31. The method according to claim 17, wherein the autosomal-dominant centronuclear myopathy is a severe or mild form of ADCNM.

32. The method according to claim 17, wherein the autosomal-dominant centronuclear myopathy is ADCNM at early or late onset.


SEQUENCE LISTING (34 sequences)

SEQ ID NO: 1 - DNA, 1782 bp, Homo sapiens
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaagcctgtctcgctgcttgagaaagccgccccccagtggtgccaaggcaaactgcaggctcatctcgtagctcaaactaacctgctccgaaatcaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagctccggaaaggcccaccagtccctccgcctcccaaacacaccccgtccaaggaagtcaagcaggagcagatcctcagcctgtttgaggacacgtttgtccctgagatcagcgtgaccaccccctcccagtttgaggccccggggcctttctcggagcaggccagtctgctggacctggactttgaccccctcccgcccgtgacgagccctgtgaaggcacccacgccctctggtcagtcaattccatgggacctctgggagcccacagagagtccagccggcagcctgccttccggggagcccagcgctgccgagggcacctttgctgtgtcctggcccagccagacggccgagccggggcctgcccaaccagcagaggcctcggaggtggcgggtgggacccaacctgcggctggagcccaggagccaggggagacggcggcaagtgaagcagcctccagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 2 - protein, 593 aa, Homo sapiens
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKPVSLLEKAAPQWCQGKLQAHLVAQTNLLRNQAEEELIKAQKVFEEMNVDLQEELPSLWNSRVGFYVNTFQSIAGLEENFHKEMSKLNQNLNDVLVGLEKQHGSNTFTVKAQPSDNAPAKGNKSPSPPDGSPAATPEIRVNHEPEPAGGATPGATLPKSPSQLRKGPPVPPPPKHTPSKEVKQEQILSLFEDTFVPEISVTTPSQFEAPGPFSEQASLLDLDFDPLPPVTSPVKAPTPSGQSIPWDLWEPTESPAGSLPSGEPSAAEGTFAVSWPSQTAEPGPAQPAEASEVAGGTQPAAGAQEPGETAASEAASSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 3 - DNA, 84 bp, Homo sapiens
atggcagagatgggcagtaaaggggtgacggcgggaaagatcgccagcaacgtgcagaagaagctcacccgcgcgcaggagaag

SEQ ID NO: 4 - DNA, 81 bp, Homo sapiens
gttctccagaagctggggaaggcagatgagaccaaggatgagcagtttgagcagtgcgtccagaatttcaacaagcagctg

SEQ ID NO: 5 - DNA, 55 bp, Homo sapiens
acggagggcacccggctgcagaaggatctccggacctacctggcctccgtcaaag

SEQ ID NO: 6 - DNA, 95 bp, Homo sapiens
ccatgcacgaggcttccaagaagctgaatgagtgtctgcaggaggtgtatgagcccgattggcccggcagggatgaggcaaacaagatcgcagag

SEQ ID NO: 7 - DNA, 96 bp, Homo sapiens
aacaacgacctgctgtggatggattaccaccagaagctggtggaccaggcgctgctgaccatggacacgtacctgggccagttccccgacatcaag

SEQ ID NO: 8 - DNA, 108 bp, Homo sapiens
tcacgcattgccaagcgggggcgcaagctggtggactacgacagtgcccggcaccactacgagtcccttcaaactgccaaaaagaaggatgaagccaaaattgccaag

SEQ ID NO: 9 - DNA, 93 bp, Homo sapiens
cctgtctcgctgcttgagaaagccgccccccagtggtgccaaggcaaactgcaggctcatctcgtagctcaaactaacctgctccgaaatcag

SEQ ID NO: 10 - DNA, 86 bp, Homo sapiens
gccgaggaggagctcatcaaagcccagaaggtgtttgaggagatgaatgtggatctgcaggaggagctgccgtccctgtggaacag

SEQ ID NO: 11 - DNA, 76 bp, Homo sapiens
ccgcgtaggtttctacgtcaacacgttccagagcatcgcgggcctggaggaaaacttccacaaggagatgagcaag

SEQ ID NO: 12 - DNA, 83 bp, Homo sapiens
ctcaaccagaacctcaatgatgtgctggtcggcctggagaagcaacacgggagcaacaccttcacggtcaaggcccagcccag

SEQ ID NO: 13 - DNA, 45 bp, Homo sapiens
aaagaaaagtaaactgttttcgcggctgcgcagaaagaagaacag

SEQ ID NO: 14 - DNA, 145 bp, Homo sapiens
tgacaacgcgcctgcaaaagggaacaagagcccttcgcctccagatggctcccctgccgccacccccgagatcagagtcaaccacgagccagagccggccggcggggccacgcccggggccaccctccccaagtccccatctcag

SEQ ID NO: 15 - DNA, 129 bp, Homo sapiens
ctccggaaaggcccaccagtccctccgcctcccaaacacaccccgtccaaggaagtcaagcaggagcagatcctcagcctgtttgaggacacgtttgtccctgagatcagcgtgaccaccccctcccag

SEQ ID NO: 16 - DNA, 108 bp, Homo sapiens
tttgaggccccggggcctttctcggagcaggccagtctgctggacctggactttgaccccctcccgcccgtgacgagccctgtgaaggcacccacgccctctggtcag

SEQ ID NO: 17 - DNA, 24 bp, Homo sapiens
tcaattccatgggacctctgggag

SEQ ID NO: 18 - DNA, 108 bp, Homo sapiens
cccacagagagtccagccggcagcctgccttccggggagcccagcgctgccgagggcacctttgctgtgtcctggcccagccagacggccgagccggggcctgcccaa

SEQ ID NO: 19 - DNA, 90 bp, Homo sapiens
ccagcagaggcctcggaggtggcgggtgggacccaacctgcggctggagcccaggagccaggggagacggcggcaagtgaagcagcctcc

SEQ ID NO: 20 - DNA, 111 bp, Homo sapiens
agctctcttcctgctgtcgtggtggagaccttcccagcaactgtgaatggcaccgtggagggcggcagtggggccgggcgcttggacctgcccccaggtttcatgttcaag

SEQ ID NO: 21 - DNA, 102 bp, Homo sapiens
gtacaggcccagcacgactacacggccactgacacagacgagctgcagctcaaggctggtgatgtggtgctggtgatccccttccagaaccctgaagagcag

SEQ ID NO: 22 - DNA, 108 bp, Homo sapiens
gatgaaggctggctcatgggcgtgaaggagagcgactggaaccagcacaaggagctggagaagtgccgtggcgtcttccccgagaacttcactgagagggtcccatga

SEQ ID NO: 23 - DNA, 809 bp, Artificial Sequence
  artificial cDNA sequence with exons 1 to 6 and 8 to 11
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagaaagaaaagtaaactgttttcgcggctgcgcagaaagaagaacag

SEQ ID NO: 24 - protein, 270 aa, Artificial Sequence
  AMINO ACID SEQUENCE corresponding to artificial cDNA sequence with exons 1 to 6 and 8 to 11
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKAEEELIKAQKVFEEMNVDLQEELPSLWNSRVGFYVNTFQSIAGLEENFHKEMSKLNQNLNDVLVGLEKQHGSNTFTVKAQPRKKSKLFSRLRRKKNS

SEQ ID NO: 25 - DNA, 1320 bp, Homo sapiens
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagccagcagaggcctcggaggtggcgggtgggacccaacctgcggctggagcccaggagccaggggagacggcggcaagtgaagcagcctccagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 26 - protein, 439 aa, Homo sapiens
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKAEEELIKAQKVFEEMNVDLQEELPSLWNSRVGFYVNTFQSIAGLEENFHKEMSKLNQNLNDVLVGLEKQHGSNTFTVKAQPSDNAPAKGNKSPSPPDGSPAATPEIRVNHEPEPAGGATPGATLPKSPSQPAEASEVAGGTQPAAGAQEPGETAASEAASSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 27 - DNA, 1275 bp, Homo sapiens
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagaaagaaaagtaaactgttttcgcggctgcgcagaaagaagaacagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 28 - protein, 424 aa, Homo sapiens
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKAEEELIKAQKVFEEMNVDLQEELPSLWNSRVGFYVNTFQSIAGLEENFHKEMSKLNQNLNDVLVGLEKQHGSNTFTVKAQPRKKSKLFSRLRRKKNSDNAPAKGNKSPSPPDGSPAATPEIRVNHEPEPAGGATPGATLPKSPSQSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 29 - DNA, 1365 bp, Homo sapiens
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagaaagaaaagtaaactgttttcgcggctgcgcagaaagaagaacagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagccagcagaggcctcggaggtggcgggtgggacccaacctgcggctggagcccaggagccaggggagacggcggcaagtgaagcagcctccagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 30 - protein, 454 aa, Homo sapiens
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKAEEELIKAQKVFEEMNVDLQEELPSLWNSRVGFYVNTFQSIAGLEENFHKEMSKLNQNLNDVLVGLEKQHGSNTFTVKAQPRKKSKLFSRLRRKKNSDNAPAKGNKSPSPPDGSPAATPEIRVNHEPEPAGGATPGATLPKSPSQPAEASEVAGGTQPAAGAQEPGETAASEAASSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 31 - DNA, 1230 bp, Artificial Sequence
  artificial cDNA sequence with exons 1 to 6; 8 to 10; 12 and 18-20 - named short isoform 9
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 32 - protein, 409 aa, Artificial Sequence
  AMINO ACID SEQUENCE corresponding to cDNA sequence with exons 1 to 6, 8 to 10, 12, and 18 to 20 - named short isoform 9
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKAEEELIKAQKVFEEMNVDLQEELPSLWNSRVGFYVNTFQSIAGLEENFHKEMSKLNQNLNDVLVGLEKQHGSNTFTVKAQPSDNAPAKGNKSPSPPDGSPAATPEIRVNHEPEPAGGATPGATLPKSPSQSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 33 - DNA, 20 bp, Artificial Sequence
  Primer BIN1
acggcgggaaagatcgccag

SEQ ID NO: 34 - DNA, 20 bp, Artificial Sequence
  Primer BIN1
ttgtgctggttccagtcgct